(19) United States
(12) Patent Application Publication
(10) Pub. No.: US 2026/0053633 A1
(43) Pub. Date: Feb. 26, 2026

(54) POROUS INTERBODIES

(71) Applicant: Alphatec Spine, Inc., Carlsbad, CA (US)

(72) Inventors: Jesse Unger, San Diego, CA (US); Scott Robinson, Encinitas, CA (US); Adam Ferreira, Encinitas, CA (US); James Murray, Cardiff, CA (US); Alec Boyle, San Diego, CA (US); Tyler Koski, Wilmette, IL (US); Graham Witherby, Dana Point, CA (US)

(21) Appl. No.: 19/271,775

(22) Filed: Jul. 16, 2025

Related U.S. Application Data
(60) Provisional application No. 63/686,730, filed on Aug. 23, 2024.

(51) Int. Cl. A61F 2/44; A61F 2/30

(52) U.S. Cl. CPC A61F 2/4455; A61F 2/30767; A61F 2002/3092

(57) ABSTRACT

In various aspects, an implant includes a body having a lattice structure, with the lattice structure extending continuously from a superior surface of the body to an inferior surface of the body. The lattice structure may define a gradient of pores, with each pore in the gradient having a plurality of vertices. Additionally, the implant may include a plurality of projections extending superiorly from each of the plurality of vertices of each pore of the gradient of pores at the superior surface of the body, with each projection of the plurality of projections having a directionality. The implant may also include one or more radiographic markers incorporated into the lattice structure, the one or more radiographic markers having a density greater than the density of the lattice structure immediately around the one or more radiographic markers and being comprised of the same material as the lattice structure.

### RELATED APPLICATION

The present application claims priority from U.S. Application No. 63/686,730, filed Aug. 23, 2024, the entirety of which is incorporated herein by reference.

### TECHNICAL FIELD

This disclosure relates generally to spinal implants and, more specifically, to porous interbody spinal implants.

### SUMMARY

Disclosed are systems, devices, and/or methods of use thereof regarding spinal implants and, more specifically, to porous interbody spinal implants. In various aspects, a surgical implant includes a body having a lattice structure, where the lattice structure extends from a superior surface of the body to an inferior surface of the body and a first lateral edge to a second lateral edge. The lattice structure defines (i) the superior surface and the inferior surface of the body, (ii) a central void of the body, (iii) a gradient of pore sizes, and (iv) a gradient of beam diameters. Both beam diameter and pore size may vary from the superior and inferior surfaces of the body and from exterior to interior surfaces of the body. The surgical implant may additionally include a plurality of superior surface projections extending superiorly from a portion of the lattice structure at the superior surface, with each of the plurality of superior surface projections having a directionality for reducing migration of the surgical implant after placement of the surgical implant during a surgical procedure. Further, the surgical implant may include one or more radiographic markers incorporated into the body.

In various aspects, an implant for use in spinal surgical procedures includes a body having a lattice structure, with the lattice structure extending continuously from a superior surface of the body to an inferior surface of the body. The lattice structure may define a gradient of pores, with each pore in the gradient having a plurality of vertices. Additionally, the implant may include a plurality of projections extending superiorly from each of the plurality of vertices of each pore of the gradient of pores at the superior surface of the body, with each projection of the plurality of projections having a directionality. The implant may also include one or more radiographic markers incorporated into the lattice structure, the one or more radiographic markers having a density greater than the density of the lattice structure immediately around the one or more radiographic markers and being comprised of the same material as the lattice structure.

In various aspects, a spinal implant device includes a body having a lattice structure and a plurality of pores defined by the lattice structure. A first group of pores may be defined at a superior surface of the body, with each pore of the first group of pores defined by a plurality of vertices. The spinal implant may also include a plurality of projections extending superiorly from at least some of the plurality of vertices of the first group of pores, with each projection of the plurality of projections having a directionality to reduce spinal implant migration.

In various aspects, a method of manufacturing a surgical implant includes forming a first portion of a lattice structure, the first portion of the lattice structure defining a first plurality of pores having a first average pore size. The method may also include forming a second portion of the lattice structure over and continuous with the first portion of the lattice structure, the second portion of the lattice structure defining a second plurality of pores having a second average pore size different from the first average pore size. Further, the method may include forming a third portion of the lattice structure over and continuous with the second portion of the lattice structure such that the first, second, and third portions of the lattice structure are continuous with each other, the third portion of the lattice structure defining a third plurality of pores having a third average pore size different from the second average pore size, where the second portion may be more radiolucent than the first portion. Still further, the method may include forming one or more radiographic markers within and continuous with the lattice structure of the second portion.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 illustrates a top or superior surface view of an implant;

FIG. 2A illustrates an anterior view of the implant of FIG. 1 and FIG. 2B illustrates an anterior perspective view of the implant of FIG. 2A;

FIG. 3 illustrates a superior, cross-sectional view of the implant of FIG. 1;

FIG. 4 illustrates a close-up anterior view of a lateral edge or side of the implant of FIGS. 1 and 2A;

FIG. 5 illustrates a lateral end view of the implant of FIGS. 1 through 4;

FIG. 6 illustrates a radiographic image of the implant of FIGS. 1 through 5 placed within a body of a subject through a surgical procedure;

FIG. 7 illustrates a top or superior surface view of another embodiment of an implant;

FIG. 8A illustrates an anterior view of the implant of FIG. 7 and FIG. 8B illustrates an anterior perspective view of the implant of FIG. 8A;

FIG. 9 illustrates the implant of FIG. 7 with a close-up view of the superior surface of the body of the implant;

FIGS. 10A-10C illustrate, respectively, a close-up view of the superior or inferior surface of the implant of FIG. 7 (FIG. 10A), another view of the superior or inferior surface of the implant of FIG. 7 (FIG. 10B), and how the design of the superior and inferior surface can facilitate rotation of the implant of FIG. 7 within the intervertebral disc space (FIG. 10C);

FIG. 11 illustrates a lateral end view of the implant of FIGS. 7 through 10B;

FIG. 12 illustrates a radiographic image of the implant of FIGS. 7 through 11 placed within a body of a subject through a surgical procedure.

FIG. 13 illustrates a top or superior surface view of another embodiment of an implant;

FIG. 14A illustrates an anterior view of the implant of FIG. 13 and FIG. 14B illustrates an anterior perspective view of the implant of FIG. 14A;

FIG. 15 illustrates a lateral end view of the implant of FIGS. 13 through 14B;

FIG. 16 illustrates a top or superior surface view of another embodiment of an implant;

FIG. 17A illustrates an anterior view of the implant of FIG. 16 and FIG. 17B illustrates an anterior perspective view of the implant of FIG. 17A;

FIG. 18 illustrates a lateral end view of the implant of FIGS. 16 through 17B;

FIGS. 19A through 19D illustrate the implants of FIGS. 1, 7, 13, and 16;

FIG. 20 illustrates additional views of the implant of FIGS. 7 through 11; and

FIG. 21 is a flowchart of an example method of manufacturing any one of the implants from FIGS. 1 through 19D.

### DETAILED DESCRIPTION

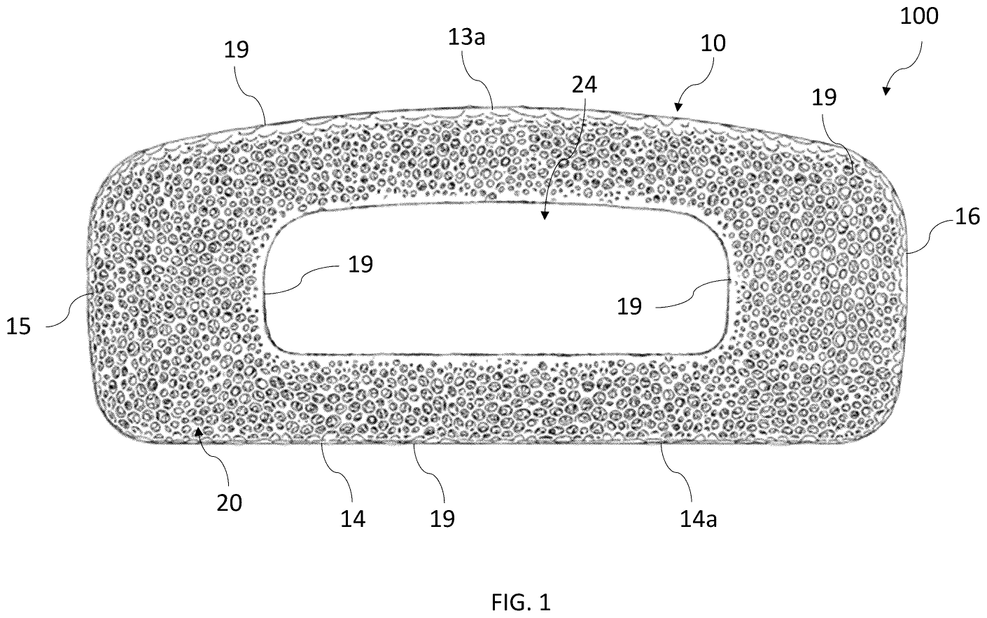

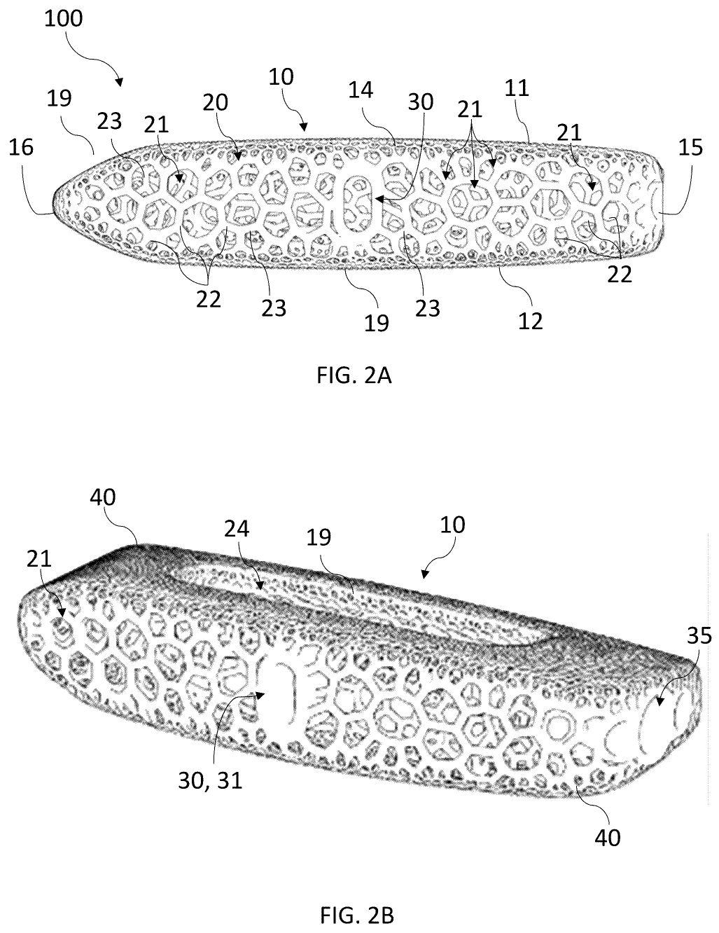

FIGS. 1 through 4 illustrate one embodiment of an implant 100. The implant 100 may be a spinal interbody implant for placement in a spinal column of a body of a patient or subject during a surgical procedure. Implant 100 may have features that are desirable in standard lumbar interbody fusion procedures. The implant 100 includes a body 10 that has a lattice structure 20, where the body 10 and/or the lattice structure 20 defines a central void 24. The body 10 may be formed from and defined by the lattice structure 20. Specifically, the lattice structure 20 extends from a superior surface 11 to an inferior surface 12 of the body 10, and from a first lateral edge 15 to a second lateral edge 16 (FIG. 2A). Additionally, the lattice structure 20 extends from an anterior region 13 including an anterior face 13a to a posterior region 14 including a posterior face 14a.

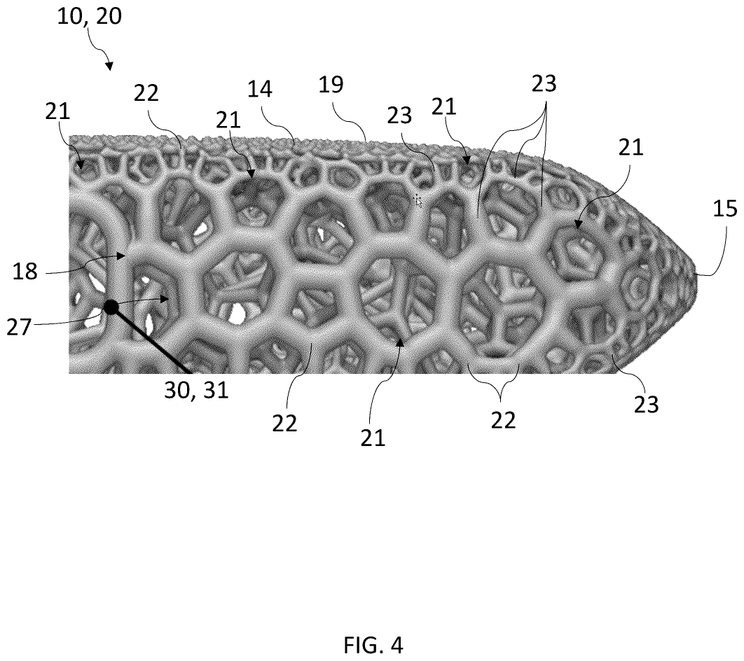

The lattice structure 20 may define the superior surface 11, the inferior surface 12, the first lateral edge 15, the second lateral edge 16, the anterior region 13, the posterior region 14, and the central void 24. That is, the lattice structure 20 defines the boundaries of the body 10. Additionally, referring briefly to FIGS. 2A and 4, the lattice structure 20 defines a plurality of pores 21. Pores may be any suitable polygonal shape. In some embodiments, each pore 21 of the plurality of pores 21 is defined by a plurality of beams 23, and a plurality of vertices 22 where beams 23 meet. The number of vertices 22 and beams 23 defining each individual pore 21 may vary. For example, some pores 21 are defined by five (5) beams 23 joined with each other through five (5) vertices 22. Alternatively, some pores 21 may be defined by six (6) beams 23 joined with each other through six (6) vertices 22, and some pores 21 may be defined by seven (7) beams 23 joined with each other through seven (7) vertices 22. The pores 21 may be defined by any number of beams 23 and vertices 22. Beams 23 and vertices 22 defining a first pore 21 may be shared and define a second, adjacent pore 21. In some embodiments, at least some of the pores 21 may be formed more as a web structure without defined beams 23 or vertices 22, such as is seen in FIG. 1.

The lattice structure 20 may be a stochastic or a substantially random lattice structure 20. In some embodiments, the lattice structure 20 is a mesh structure. The lattice structure 20 may define a gradient of pore sizes as well as a gradient of beam diameters. As seen most clearly in FIG. 4, a size of pores 21 at the superior surface 11 may be smaller than a size of pores 21 in a middle region 18, between the superior surface 11 and the inferior surface 12. The size of pores 21 may become smaller again at the inferior surface 12. That is, the pore size may be a smaller pore size at the exterior edges of the body, with a larger pore size at the interior of the body, and a gradient of sizes between the smaller exterior pore size and the larger interior pore size.

The size of pores 21 at the superior surface 11 and/or the inferior surface 12 may range from about 100 microns to about 1,000 microns, such as 200, 300, 350, 400, 450, 500, 550, 600, 650, 700, 750, 800, 850, 900, 950 microns, or a size within a range defined by any two of the foregoing values. Pores 21 having this size may be referred to as “micro pores.” The size of pores 21 in the middle region 18 may range from about 1,000 microns to about 8,000 microns, such as 1,500, 2,000, 2,500, 3,000, 3,500, 4,000, 4,500, 5,000, 5,500, 6,000, 6,500, 7,000, 7,500 microns, or a size within a range defined by any two of the foregoing values. Pores 21 having this size may be referred to as “macro pores.” The size of pores 21 at exterior regions 19 may be larger than a size of pores 21 at interior regions 27 of the lattice structure 20. Pores may be sized and shaped to allow optimal bone in-growth, as well as maximize the amount of bone graft material that can be packed into the implant to improve patient outcomes.

Additionally, the gradient of beam diameters may follow or correspond to the gradient of pore sizes. Specifically, a diameter of the beams 23 at the superior surface 11 and/or the inferior surface 12 may be smaller than a diameter of the beams 23 in the middle region 18. The defined gradient of pore sizes allows regions of a first pore size to transition to regions of a second pore size in a continuous and seamless manner. Specifically, as seen most clearly in FIG. 4, the pores 21 at the superior surface 11 share beams 23 with pores 21 in the middle region 18. Though the beam 23 diameter may change as the pores 21 transition from the superior surface 11 to the middle region 18, the transition between pore sizes is continuous (i.e., no clear boundaries or layers between the differing pore sizes). This increases the overall integrity of the implant 100.

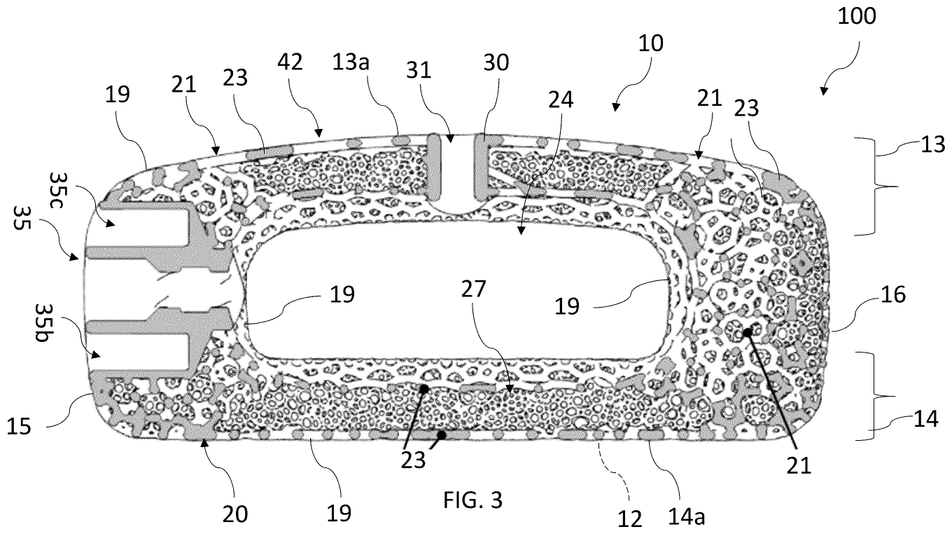

The body 10 and/or the lattice structure 20 includes exterior regions 19 and interior regions 27 (see FIG. 3). The exterior region 19 may correspond to boundaries of and be defined by the lattice structure 20. For example, the lattice structure 20 defines the superior surface 11 and the inferior surface 12, where the superior surface 11 and the inferior surface 12 correspond to an exterior region 19 of the implant 100. Similarly, the lattice structure 20 defines the central void 24 and boundaries of the central void 24 may correspond to an exterior region 19. Likewise, areas or regions between or “inside” the superior surface 11, the exterior regions 19, and the inferior surface 12 may correspond to interior regions 27. In some embodiments, the exterior regions 19 correspond to surfaces of the body 10 that may be directly interfacing with patient tissues, while interior regions 27 are not directly touching patient tissues. Similarly, exterior regions 19 may be touched or handled by the clinician, while the interior regions 27 are not accessible for direct contact by a clinician.

In some embodiments, the gradient of pore sizes and beam diameters is also present from exterior regions 19 to interior regions 27. That is, a size of pores 21 at exterior regions 19 may be larger than a size of pores 21 at interior regions 27 of the lattice structure 20 and/or the body 10. Similarly, the beam diameters at exterior regions 19 may be larger than beam diameters at interior regions 27. Alternatively, the size of pores 21 at exterior regions 19 may be smaller than a size of pores 21 at interior regions 27 of the lattice structure 20 and/or the body 10. Or, the pores and/or beam diameters may be the same size throughout the body.

The variability and gradient of pore sizes and beam diameters achieves varying zones of stiffness between the superior and/or inferior surfaces 11, 12 as compared to an interior zone or region 27 of the body 10 and/or the lattice structure 20. This may result in the superior and/or inferior surfaces 11, 12 being stiffer than the interior regions 27; alternatively, the superior and/or inferior surfaces 11, 12 may be less stiff than the interior regions 27. Additionally, the variability of pore sizes and beam diameters achieves varying zones of stiffness between the exterior 19 of the body 10 as compared to an interior zone 27 of the body 10.

The variability and gradient of pore sizes and beam diameters may also be used to achieve a desired overall stiffness of the body 10, which stiffness may be adjusted based on patient-specific bone density. For example, in some embodiments, a bone density scan may be performed prior to surgery, and, based on those results, an interbody of a given overall stiffness may be selected or one may be manufactured to spec. It may be desirable to have an interbody with an overall stiffness that roughly matches the bone density of the patient or that is within a certain standard deviation of the patient's bone density. This same bone density information may be used to select or modify the stiffness of the superior and/or inferior surfaces 11, 12.

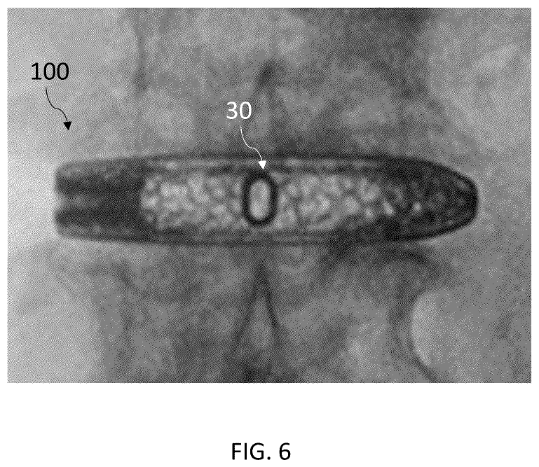

The body 10 and/or the lattice structure 20 additionally includes one or more radiographic markers 30 and an inserter engagement portion 35. The one or more radiographic markers 30 allow practitioners to ensure proper placement and implantation of the implant 100 in a body of a subject (also referred to as “patient anatomy”). Specifically, the one or more radiographic markers 30 clearly mark the mid-line of the implant 100 when the implant 100 is properly positioned within the body of the subject, thereby providing a clear visualization that the implant has been properly positioned. FIG. 6 illustrates a radiographic image of the implant 100 of FIGS. 1 through 5 placed within patient anatomy through a surgical procedure. For example, the implant 100 may be placed within patient anatomy during a spinal surgical procedure, and radiographic markers 30 may align with a patient's spinous process or other desired anatomy. A radiographic marker 30 may be incorporated into the anterior region 13 of the lattice structure 20 and/or the body 10. The one or more radiographic markers 30 may utilize improved radiolucency, relative to the rest of the lattice structure 20, as a marker.

As seen most clearly in FIG. 3, the one or more radiographic markers 30 may include a hollow channel 31 defined within the lattice structure 20 in the anterior region 13 of the implant 100. This hollow channel 31 defines a negative space (i.e., a space wherein the lattice structure of the body does not extend), which may be surrounded by a dense material, such as the material that comprises the lattice structure 20 or a different material, to provide a more visible contrast in radiographic images. The hollow channel 31 may extend internally from an exterior surface or region 19 of the body 10 towards an interior 27 of the body 10. The hollow channel 31 may be circular, elliptical, square, rectangular, or oblong. If the hollow channel 31 is elliptical, rectangular, or oblong, it may be positioned horizontally (e.g., along lateral edge 15 to lateral edge 16) or vertically (e.g., along superior surface 11 to inferior surface 12) within the body.

The one or more radiographic markers 30 may have a depth and be formed from the same material as the body 10. In some embodiments, the one or more radiographic markers 30 are embedded within the lattice structure 20 and characterized by portions of the lattice structure 20 being denser than the body 10. That is, the one or more radiographic markers 30 may be characterized by multiple layers of material laid on top of each other, thereby creating denser areas of material in association with the one or more radiographic markers 30. The one or more radiographic markers 30 may be printable within and supported by the lattice structure 20. The one or more radiographic markers 30 may be formed from the same material as the lattice structure 20. For example, the one or more radiographic markers 30 and the lattice structure 20 may be formed from titanium alloy (grade 23 titanium alloy or any other suitable titanium alloy or any other suitable material). The one or more radiographic markers 30 may be formed from a different material than the lattice structure 20 and may comprise one or more pins of a distinct material, such as a material that is more radiopaque than the material that comprises the lattice structure 20, inserted into voids formed in the lattice structure 20.

In some configurations, the body 10 may have an interfixated design, such that screws or other fasteners may extend through the anterior region 13 of the body 10 into adjacent bone when the implant 100 is implanted within patient anatomy. The body may include channels to receive screws or fasteners, the channels extending from the anterior region 13 to exit at a superior surface 11 and/or inferior surface 12, and/or through a portion of the anterior region 13. In other configurations, the body may receive screws or other fasteners for interfixation without providing specific channels.

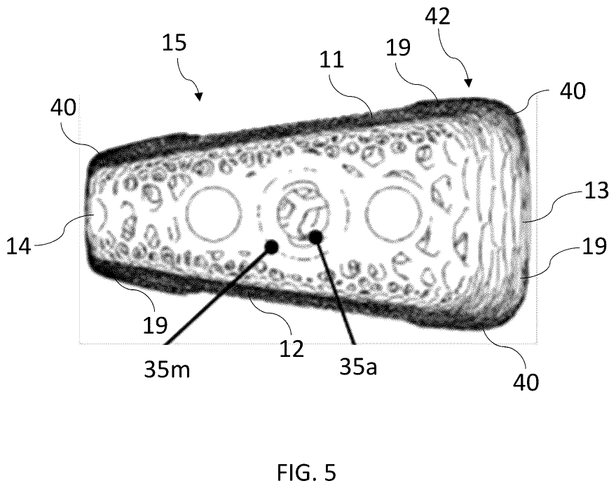

The inserter engagement 35 (FIG. 3) is visible at the lateral edge 15 of the implant 100, which may correspond to a proximal end of the body 10. In some embodiments, the inserter engagement portion 35 includes or defines an interior space accessible through an elongated window in a proximal end of the body 10, where the elongated window includes a plurality of raised surface features configured to engage with a portion of the inserter, and where the interior space is cylindrical. FIG. 5 illustrates a lateral end view of the implant 100 of FIGS. 1 through 4. The inserter engagement 35 may include one or more threadings, such as an Acme threading 35a and/or an M-5 threading 35m. The inserter engagement 35 may also include additional voids 35b,c to receive and engage with an insertion instrument for case in placement and alignment of the insertion instrument.

Also illustrated in FIG. 5 is the generally rimless 42 nature or configuration of the body 10. Specifically, the body 10 includes rounded or convex edges 40 that define the exterior 19 of the body 10. For example, the lateral edges 15, 16 may be rounded or convex 40. Rounded shapes may reduce point loading as well as subsidence.

Additionally, the anterior region 13 may define a convex edge 40. In this way, the body 10 is rimless 42. Put another way, the lattice structure 20 defines the exterior regions 19 of the body 10 through the continuous pore structure also defined by the lattice structure 20, eliminating any need for an additional rim or boundary to be applied to the body 10. The rimless 42 configuration of the body 10 improves the overall radiolucency of the implant 100, such that a dark line or border/rim of the implant 100 is lessened in radiographic images (see FIG. 6). Also visible in FIG. 5 is tapering of the body 10 towards the posterior region 14, such that the posterior region 14 of the body 10 is narrower than the anterior region 13 of the body 10.

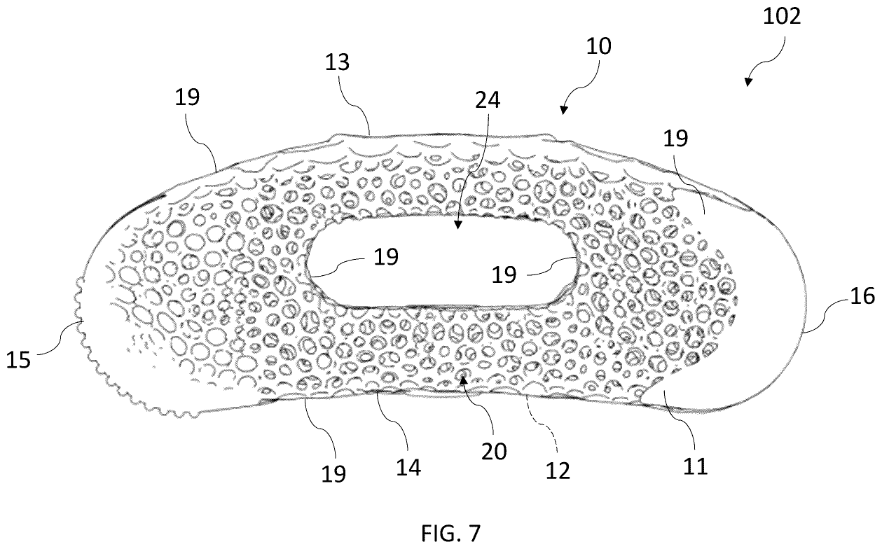

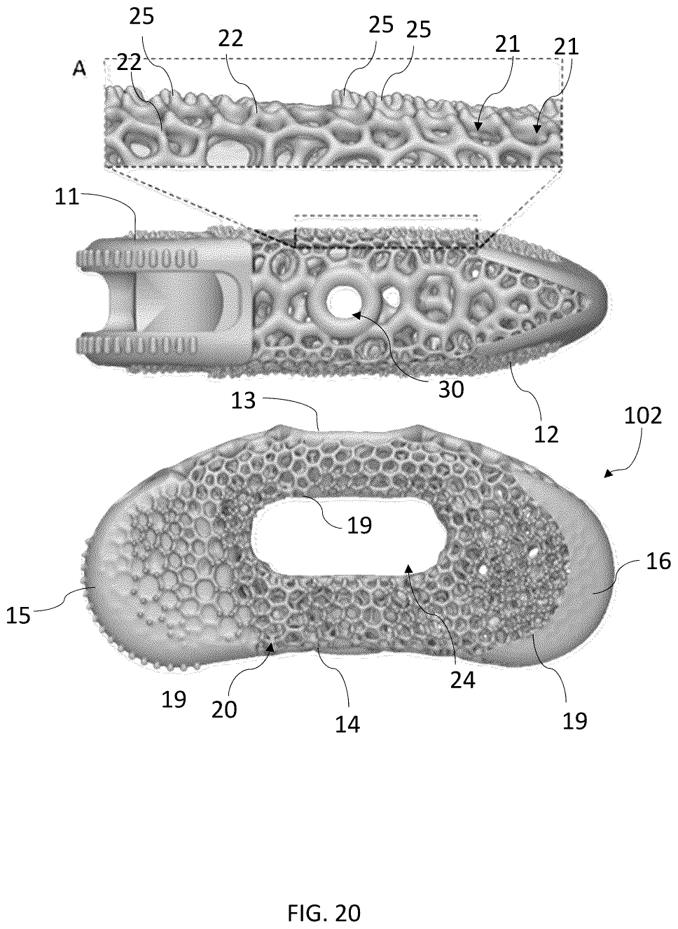

FIGS. 7 through 9 illustrate another embodiment of an implant 102 and FIG. 20 illustrates additional views of the implant 102. The implant 102 shares similar structures with the implant 100, so like reference numerals will be used to refer to like elements and structures. Implant 102 may have features that are desirable in standard transforaminal lumbar interbody fusion spinal fusion procedures. Similar to the implant 100, the implant 102 includes a body 10 that has a lattice structure 20, where the body 10 and/or the lattice structure 20 defines a central void 24. The body 10 may be formed from and defined by the lattice structure 20. Specifically, the lattice structure 20 extends from a superior surface 11 of the body 10 to an inferior surface 12 of the body 10, and from a first lateral edge 15 to a second lateral edge 16. Additionally, the lattice structure 20 extends from an anterior region 13 to a posterior region 14.

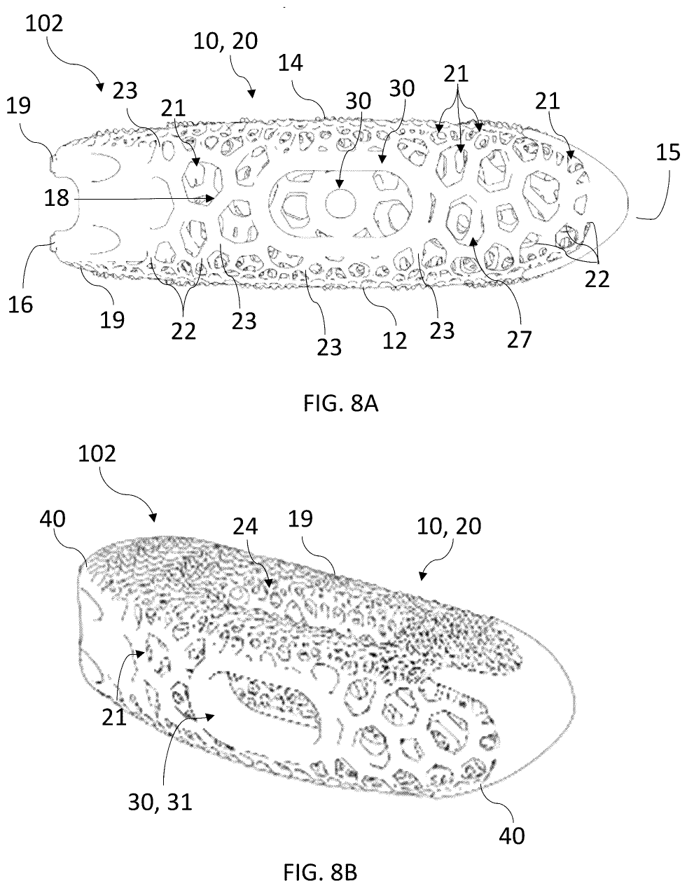

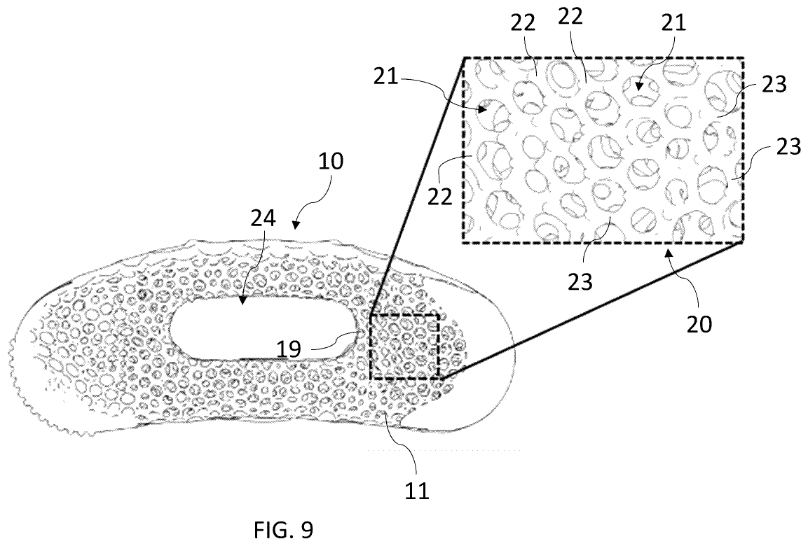

The lattice structure 20 may define the superior surface 11, the inferior surface 12, the first lateral edge 15, the second lateral edge 16, the anterior region 13, the posterior region 14, and the central void 24. That is, the lattice structure 20 defines the boundaries of the body 10. Additionally, referring briefly to FIGS. 8A and 9, the lattice structure 20 defines a plurality of pores 21. Specifically, each pore 21 of the plurality of pores 21 is defined by a plurality of vertices 22 and a plurality of beams 23. The number of vertices 22 and beams 23 defining each individual pore 21 may vary. Each pore 21 may be defined by four (4), five (5), six (6), seven (7), or more beams 23 and vertices 22. In general, the number of beams 23 defining the pores 21 corresponds to the number of vertices 22 defining the pores 21. In some embodiments, at least some of the pores 21 may be formed more as a web structure without defined beams 23 or vertices 22, such as is seen in FIG. 9.

The lattice structure 20 may be a stochastic or substantially random lattice structure 20. In some embodiments, the lattice structure 20 is a mesh structure. As with the implant 100, the lattice structure 20 may define a gradient of pore sizes as well as a gradient of beam diameters. As seen most clearly in FIG. 8A, a size of pores 21 at the superior surface 11 may be smaller than a size of pores 21 in a middle region 18, between the superior surface 11 and the inferior surface 12. The size of pores 21 may become smaller again at the inferior surface 12. As each pore 21 is defined by a plurality of beams 23 and a plurality of vertices 22, and these beams 23 and vertices 22 are shared between pores 21, the transition between pore sizes is substantially continuous and seamless.

The size of pores 21 at the superior surface 11 and/or the inferior surface 12 may range from about 100 microns to about 1,000 microns, such as 200, 300, 350, 400, 450, 500, 550, 600, 650, 700, 750, 800, 850, 900, 950 microns, or a size within a range defined by any two of the foregoing values. Pores 21 having this size may be referred to as “micro pores.” The size of pores 21 in the middle region 18 may range from about 1,000 microns to about 8,000 microns, such as 1,500, 2,000, 2,500, 3,000, 3,500, 4,000, 4,500, 5,000, 5,500, 6,000, 6,500, 7,000, 7,500 microns, or a size within a range defined by any two of the foregoing values. Pores 21 having this size may be referred to as “macro pores.” The size of pores 21 at exterior regions 19 may be larger than the size of pores 21 at interior regions 27 of the lattice structure 20.

Additionally, in some embodiments, the gradient of beam diameters follows or corresponds to the gradient of pore sizes. Specifically, a diameter of the beams 23 at the superior surface 11 and/or the inferior surface 12 may be smaller or narrower than a diameter of the beams 23 in the middle region 18. The defined gradient of pore sizes allows regions of a first pore size to transition to regions of a second pore size in a continuous and seamless manner. Specifically, as seen most clearly in FIG. 8A, the pores 21 at the superior surface 11 share beams 23 with pores 21 in the middle region 18. Though the beam 23 diameter may change as the pores 21 transition from the superior surface 11 to the middle region 18, the transition between pore sizes is continuous (i.e., no clear boundaries or layers between the differing pore sizes). This increases the overall integrity of the implant 100.

The body 10 and/or the lattice structure 20 includes exterior regions 19 and interior regions 27. The exterior region 19 may correspond to boundaries of the lattice structure 20. For example, the lattice structure 20 defines the superior surface 11 and the inferior surface 12, where the superior surface 11 and the inferior surface 12 correspond to an exterior region or surface 19 of the implant 100. Similarly, the lattice structure 20 defines the central void 24 and boundaries of the central void 24 may correspond to exterior regions or surfaces 19. Likewise, areas or regions between or “inside” the superior surface 11, the exterior regions 19, and the inferior surface 12 may correspond to interior regions 27. In some embodiments, the exterior regions 19 correspond to surfaces of the body 10 that a user or practitioner may touch, while interior regions 27 are not tactilely accessible to the user or practitioner.

The gradient of pore sizes and beam diameters is also present from exterior regions 19 to interior regions 27. That is, the size of pores 21 at exterior regions 19 may be larger than the size of pores 21 at interior regions 27 of the lattice structure 20 and/or the body 10. Similarly, the beam diameters at exterior regions 19 may be larger than beam diameters at interior regions 27.

The variability and gradient of pore sizes and beam diameters achieves varying zones of stiffness between the superior and/or inferior surfaces 11, 12 as compared to an interior zone or region 27 of the body 10 and/or the lattice structure 20. This may result in the superior and/or inferior surfaces 11, 12 being more stiff than the interior regions 27; alternatively, the superior and/or inferior surfaces 11, 12 may be less stiff than the interior regions 27. Additionally, the variability of pore sizes and beam diameters achieves varying zones of stiffness between the exterior 19 of the body 10 as compared to an interior zone 27 of the body 10.

The variability and gradient of pore sizes and beam diameters may also be used to achieve a desired overall stiffness of the body 10, which stiffness may be adjusted based on patient-specific bone density. For example, in some embodiments, a bone density scan may be performed prior to surgery, and, based on those results, an interbody of a given overall stiffness may be selected or one may be manufactured to specifications (i.e., manufactured to match the scan). It may be desirable to have an interbody with an overall stiffness that roughly matches the bone density of the patient or that is within a certain standard deviation of the patient's bone density. This same bone density information may be used to select or modify the stiffness of the superior and/or inferior surfaces 11, 12.

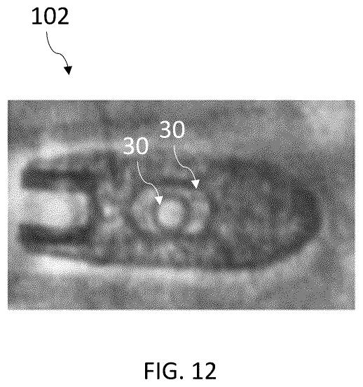

The body 10 and/or the lattice structure 20 additionally includes one or more radiographic markers 30. The one or more radiographic markers 30 allow practitioners to ensure proper placement and implantation of the implant 102 in patient anatomy. Specifically, the one or more radiographic markers 30 clearly mark the mid-line of the implant 102 when the implant 102 is properly positioned within the patient anatomy, thereby providing a clear visualization that the implant 102 has been properly positioned. FIG. 12 illustrates a radiographic image of the implant 102 of FIGS. 7 through 9 placed within a body of a subject through a surgical procedure. For example, the implant 102 may be placed within patient anatomy during a spinal surgical procedure, and radiographic markers 30 may align with a patient's spinous process or other desired anatomy. A radiographic marker 30 may be incorporated into the anterior region 13 and/or the posterior region 14 of the lattice structure 20 and/or the body 10. The one or more radiographic markers 30 may utilize improved radiolucency, relative to the rest of the lattice structure 20, as a marker. Radiographic markers 30 may comprise a portion of void space surrounded by a dense material, such as the material that comprises the lattice structure 20 or a different material, to provide a more visible contrast in radiographic images.

The one or more radiographic markers 30 may have a depth and be formed from the same material as the body 10. In some embodiments, the one or more radiographic markers 30 are embedded within the lattice structure 20 and characterized by portions of the lattice structure 20 being denser than the body 10. That is, the one or more radiographic markers 30 may be characterized by multiple layers of material laid on top of each other, thereby creating denser areas of material in association with the one or more radiographic markers 30. The one or more radiographic markers 30 may be printable within and supported by the lattice structure 20. The one or more radiographic markers 30 may be formed from the same material as the lattice structure 20. For example, the one or more radiographic markers 30 and the lattice structure 20 may be formed from grade 23 titanium alloy. The one or more radiographic markers 30 may be formed from a different material than the lattice structure 20 and may comprise one or more pins of a distinct material, such as a material that is more radiopaque than the material that comprises the lattice structure 20, inserted into voids formed in the lattice structure 20.

As seen most clearly in FIGS. 8A and 8B, the one or more radiographic markers 30 may include a hollow channel 31 defined within the lattice structure 20 in the anterior region 13 of the implant 102. The hollow channel 31 may extend internally from an exterior surface or region 19 at the anterior region 13 of the body 10 towards an interior 27 of the body 10. In some embodiments, the one or more radiographic markers 30 include two (2) radiographic markers 30, where a first radiographic marker 30 includes a first hollow channel 31 and a second radiographic marker 30 includes a second hollow channel 31 (see FIG. 8A). The second hollow channel 31 may also extend from an exterior 19 of the body 10 towards an interior 27 of the body 10. The second hollow channel 31 may be configured to align visually with the first hollow channel 31 as an indication of desired and proper positioning of the implant 102 within patient anatomy, as seen in FIG. 12. That is, the second hollow channel 31 may appear within the first hollow channel when the implant is positioned properly.

The hollow channels 31 may be circular, elliptical, square, rectangular, oblong, or any desired shape. If the first and/or second channels 31 are elliptical, rectangular, or oblong, they are positioned horizontally (e.g., along lateral edge 15 to lateral edge 16) or vertically (e.g., along superior surface 11 to inferior surface 12) within the body. In some embodiments, the first hollow channel 31 may be circular and the second hollow channel 31 may be non-circular or vice versa.

In some configurations, the body 10 may have an interfixated design, such that screws or other fasteners may extend through the anterior region 13 of the body 10 into adjacent bone when the implant 100 is implanted within patient anatomy. The body may include channels to receive screws or fasteners, the channels extending from the anterior region 13 to exit at a superior surface 11 and/or inferior surface 12, and/or through a portion of the anterior region 13. In other configurations, the body may receive screws or other fasteners for interfixation without providing specific channels.

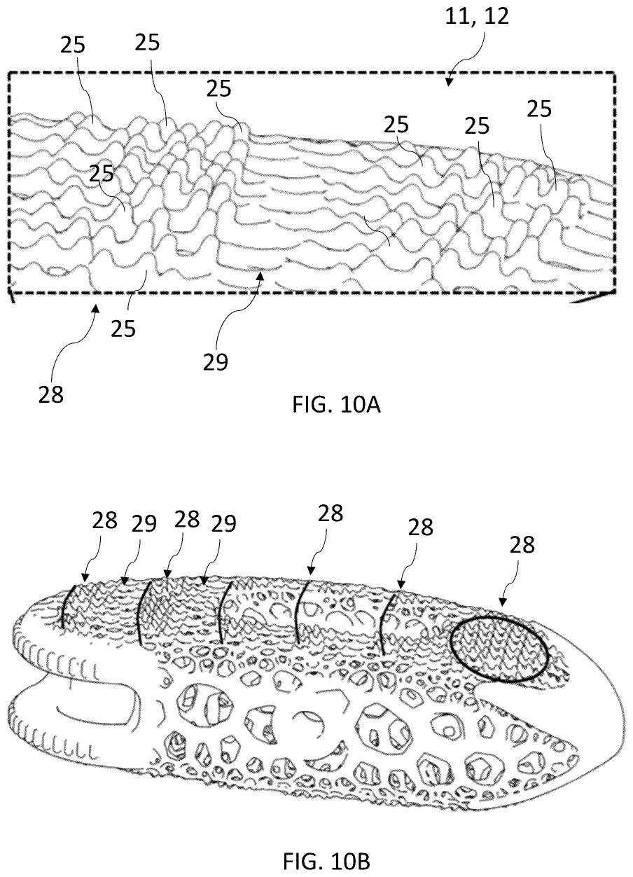

FIG. 10A illustrates a close-up view of the superior or inferior surface 11, 12 and FIG. 10B illustrates another view of the superior or inferior surface 11, 12 of the implant 102 of FIG. 7. Specifically, illustrated are a plurality of surface projections 25 that may extend from portions of the superior surface 11 and/or the inferior surface 12. For example, the plurality of surface projections 25 may extend from an individual vertex of the vertices 22 defining the pores 21 and may at least partially define the superior surface 11 and/or the inferior surface 12. The surface projections 25 are also illustrated in FIG. 20. In some embodiments, the surface projections 25 are grouped into bands 28, leaving empty bands 29 about the superior and/or inferior surfaces 11, 12. For example, as illustrated in FIG. 10B, empty bands 29 may be shaped in a curve to facilitate rotation of the implant 102 into place. Similarly, bands 28 of the surface projections 25 may be curved or otherwise shaped to facilitate rotation and placement of the implant 102.

In some embodiments, the surface projections 25 may be grouped in bands 28 disposed radially or concentrically about the central void 24. The grouped bands 28 may extend from the central void 24 to a first boundary of the body 10. In some embodiments, the surface projections 25 may be grouped in bands 28 disposed radially or concentrically about a proximal end of the body 10 and extend to a distal end of the body 10. In some embodiments, the surface projections 25 are grouped into a plurality of bands 28 extending across the superior and/or inferior surfaces 11, 12 from the first lateral edge 15 to the second lateral edge 16.

In some embodiments, the surface projections 25 may be grouped in bands 28 radiating concentrically from a distal end of the body 10 and extending toward a proximal end of the body 10. In some embodiments, bands 28 form arcs whose center is located at about or near the distal end of the body 10, such as at pivot area 32. Pivot area 32 may be comprised of a plurality of projections 25 that may or may not exhibit any particular directionality. They may be designed to simply inhibit or resist movement of the distal end of the body 10 while within the intervertebral disc space, which may facilitate rotation of the body 10 into a desired position and/or orientation within the intervertebral disc space.

FIG. 10C illustrates how the bands 28 formed by the plurality of projections 25 can facilitate a rotation of the body 10 about the pivot area 32. This feature allows a surgeon to insert the implant 102 into an intervertebral disc space with the implant 102 aligned along a direction of insertion where the direction of insertion may define a line having an angle relative to the width of the intervertebral disc space. The surgeon may continue to advance the implant 102 until the distal end is placed or positioned in a desirable location toward an anterior region of the intervertebral disc space with the distal end positioned on one side of that anterior region. The plurality of projections 25 found in the pivot region 32 may then allow a surgeon to move the proximal end of the implant 102 into the anterior region by rotating the implant 102, which rotation is further enhanced with the use of an inserter capable of selectively holding the implant 102 in either a locked orientation relative to the inserter—which facilitates implant insertion—or an articulating orientation relative to the inserter—which facilitates rotation of the implant 102 about the pivot area 32.

The surface projections 25 may extend superiorly or inferiorly, respectively, with a directionality. The directionality of the surface projections 25 may reduce migration of the implant 102 after placement of the implant 102 during a surgical procedure. The directionality of the surface projections 25 at the superior surface 11 may match or correspond to a directionality of the surface projections 25 at the inferior surface 12. In other configurations, the surface projections 25 do not have directionality.

Individual projections 25 may extend upwards or downwards (i.e., extend superiorly or inferiorly, respectively) from an individual vertex of the vertices 22 of the pores 21. In some embodiments, the surface projections 25 may exhibit varying heights. In one configuration, taller surface projections 25 are located near the central void 24 and shorter surface projections 25 are located toward the first and second lateral edges 15, 16. Though not illustrated, the body 10 may additionally include a plurality of teeth disposed about edges of the superior surface 11 of the body 10, with the plurality of teeth being disposed along a line of curvature to facilitate rotational placement of the implant 102 during a surgical procedure.

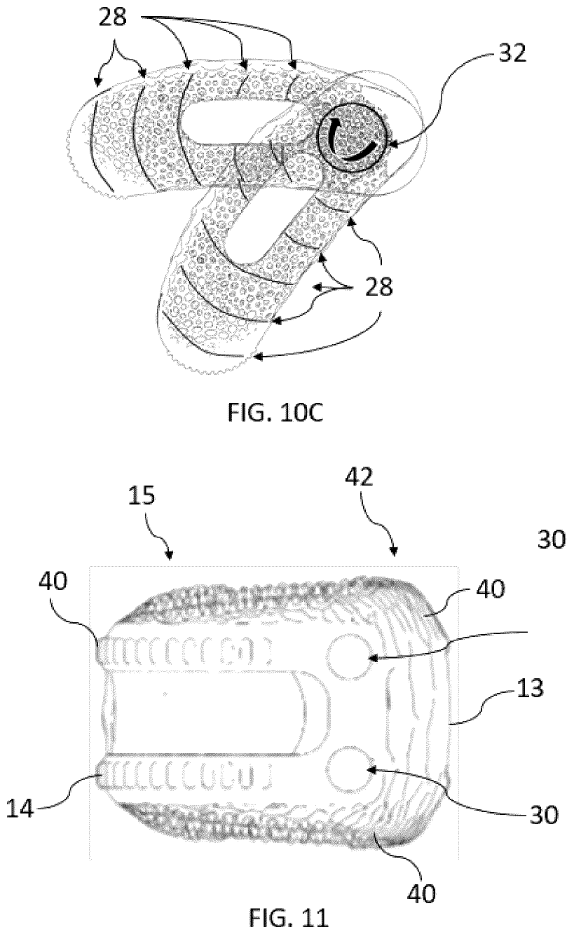

FIG. 11 illustrates a lateral end view of the implant 102 of FIGS. 7 through 10. Illustrated in FIG. 11 is the generally rimless 42 nature of the body 10. Specifically, the body 10 includes rounded or convex edges 40 that at least partially define the exterior 19 of the body 10. In this way, the body 10 is rimless 42. Put another way, the lattice structure 20 defines the exterior regions 19 of the body 10 through the continuous pore structure also defined by the lattice structure 20, eliminating any need for an additional rim or boundary to be applied to the body 10. The rimless 42 configuration of the body 10 improves the overall radiolucency of the implant 100, such that a dark line or border is missing from the implant 102 in radiographic images (see FIG. 12). This aids in visual confirmation that the implant 102 has been properly positioned within patient anatomy. The rimless body 10 also allows more graft material to be packed within the implant to improve patient outcomes.

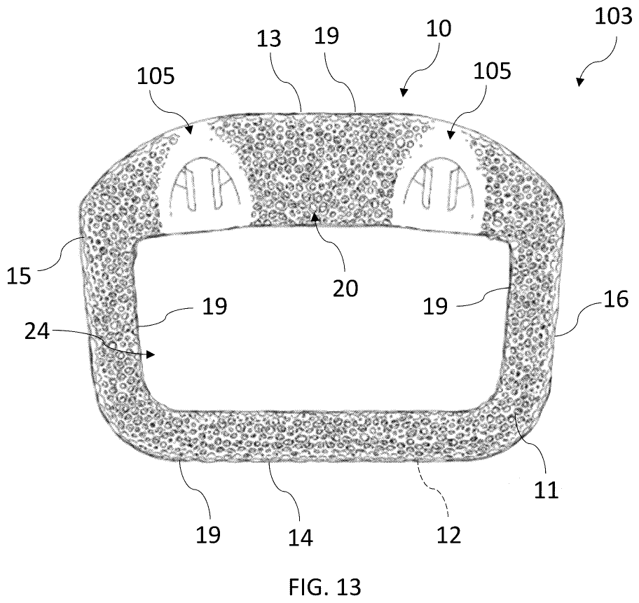

FIGS. 13 through 15 illustrate another embodiment of an implant 103. The implant 103 shares similar structures with the implant 100 and the implant 102, so like reference numerals will be used to refer to like elements and structures. Implant 103 may have features that are suitable for common ALIF spinal fusion procedures. Similar to the implants 100 and 102, the implant 103 includes a body 10 that has a lattice structure 20, where the body 10 and/or the lattice structure 20 defines a central void 24. The body 10 may be formed from and defined by the lattice structure 20. Specifically, the lattice structure 20 extends from a superior surface 11 of the body 10 to an inferior surface 12 of the body 10, and from a first lateral edge 15 to a second lateral edge 16. Additionally, the lattice structure 20 extends from an anterior region 13 to a posterior region 14.

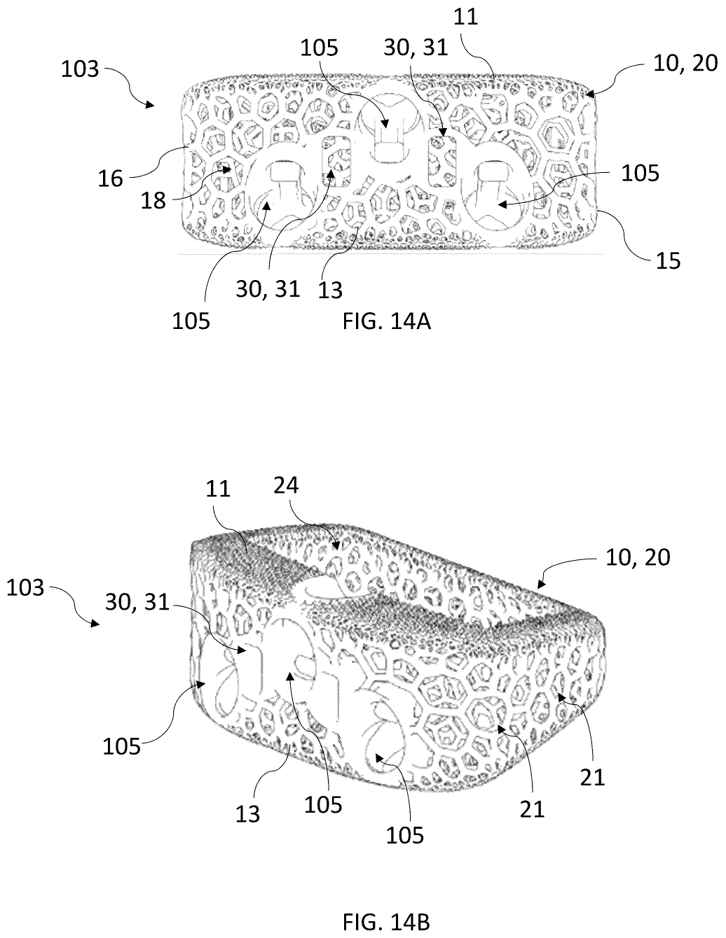

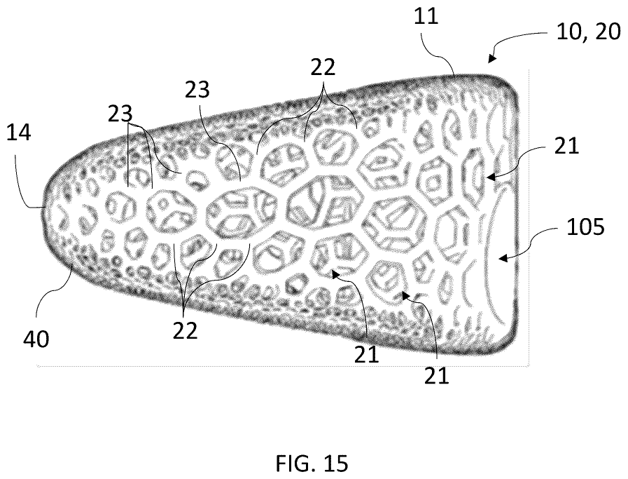

The lattice structure 20 may define the superior surface 11, the inferior surface 12, the first lateral edge 15, the second lateral edge 16, the anterior region 13, and the posterior region 14. That is, the lattice structure 20 defines the boundaries of the body 10. Additionally, referring briefly to FIGS. 14B through 15, the lattice structure 20 defines a plurality of pores 21. Specifically, each pore 21 of the plurality of pores 21 is defined by a plurality of vertices 22 and a plurality of beams 23. The number of vertices 22 and beams 23 defining each individual pore 21 may vary. Each pore 21 may be defined by four (4), five (5), six (6), seven (7), or more beams 23 and vertices 22. In general, the number of beams 23 defining the pores 21 corresponds to the number of vertices 22 defining the pores 21.

The lattice structure 20 may be a stochastic or substantially random lattice structure 20. In some embodiments, the lattice structure 20 is a mesh structure. As with the implants 100 and 102, the lattice structure 20 may define a gradient of pore sizes as well as a gradient of beam diameters. As seen most clearly in FIG. 14A, a size of pores 21 at the superior surface 11 may be smaller than a size of pores 21 in a middle region 18, between the superior surface 11 and the inferior surface 12. The size of pores 21 may become smaller again at the inferior surface 12. As each pore 21 is defined by a plurality of beams 23 and a plurality of vertices 22, and these beams 23 and vertices 22 are shared between pores 21, the transition between pore sizes is substantially continuous and seamless.

The size of pores 21 at the superior surface 11 and/or the inferior surface 12 may range from about 100 microns to about 1,000 microns, such as 200, 300, 350, 400, 450, 500, 550, 600, 650, 700, 750, 800, 850, 900, 950 microns, or a size within a range defined by any two of the foregoing values. Pores 21 having this size may be referred to as “micro pores.” The size of pores 21 in the middle region 18 may range from about 1,000 microns to about 8,000 microns, such as 1,500, 2,000, 2,500, 3,000, 3,500, 4,000, 4,500, 5,000, 5,500, 6,000, 6,500, 7,000, 7,500 microns, or a size within a range defined by any two of the foregoing values. Pores 21 having this size may be referred to as “macro pores.” The size of pores 21 at exterior regions 19 may be larger than the size of pores 21 at interior regions 27 of the lattice structure 20.

Additionally, the gradient of beam diameters follows or corresponds to the gradient of pore sizes. Specifically, a diameter of the beams 23 at the superior surface 11 and/or the inferior surface 12 may be smaller or narrower than a diameter of the beams 23 in the middle region 18. The defined gradient of pore sizes allows regions or groups of a first pore size to transition to regions or groups of a second pore size in a continuous and seamless manner. Specifically, as seen most clearly in FIG. 14A, the pores 21 at the superior surface 11 share beams 23 with pores 21 in the middle region 18. Though the beam 23 diameter may change as the pores 21 transition from the superior surface 11 to the middle region 18, the transition between pore sizes is continuous (i.e., no clear boundaries or layers between the differing pore sizes). This increases the overall integrity of the implant 103.

The body 10 and/or the lattice structure 20 includes exterior regions 19 and interior regions 27. The exterior region 19 may correspond to boundaries of the lattice structure 20. For example, the lattice structure 20 defines the superior surface 11 and the inferior surface 12, where the superior surface 11 and the inferior surface 12 correspond to an exterior region or surface 19 of the implant 100. Similarly, the lattice structure 20 defines the central void 24 and boundaries of the central void 24 may correspond to exterior regions or surfaces 19. Likewise, areas or regions between or “inside” the superior surface 11, the exterior regions 19, and the inferior surface 12 may correspond to interior regions 27. In some embodiments, the exterior regions 19 correspond to surfaces of the body 10 that a user or practitioner may touch, while interior regions 27 are not tactilely accessible to the user or practitioner.

The gradient of pore sizes and beam diameters is also present from exterior regions 19 to interior regions 27. That is, the size of pores 21 at exterior regions 19 may be larger than the size of pores 21 at interior regions 27 of the lattice structure 20 and/or the body 10. Similarly, the beam diameters at exterior regions 19 may be larger than beam diameters at interior regions 27.

The variability and gradient of pore sizes and beam diameters achieves varying zones of stiffness between the superior and/or inferior surfaces 11, 12 as compared to an interior zone or region 27 of the body 10 and/or the lattice structure 20. This may result in the superior and/or inferior surfaces 11, 12 being more stiff than the interior regions 27; alternatively, the superior and/or inferior surfaces 11, 12 may be less stiff than the interior regions 27. Additionally, the variability of pore sizes and beam diameters achieves varying zones of stiffness between the exterior 19 of the body 10 as compared to an interior zone 27 of the body 10.

As seen in FIG. 14B, the body 10 may include a plurality of surface projections 25 disposed about portions of the superior surface 11 and/or the inferior surface 12. As before, the surface projections 25 may reduce migration of the implant 103 after implantation and placement of the implant 103 in patient anatomy, such as in a spinal column of a patient.

The body 10 and/or the lattice structure 20 additionally includes one or more radiographic markers 30. The one or more radiographic markers 30 allow practitioners to ensure proper placement and implantation of the implant 103 in a body of a subject. Specifically, the one or more radiographic markers 30 clearly mark the mid-line of the implant 103 when the implant 103 is properly positioned within the body of the subject, thereby providing a clear visualization that the implant 103 has been properly positioned. For example, the implant 103 may be placed within patient anatomy during a spinal surgical procedure. A radiographic marker 30 may be incorporated into the anterior region 13 and/or the posterior region 14 of the lattice structure 20 and/or the body 10. The one or more radiographic markers 30 may utilize improved radiolucency, relative to the rest of the lattice structure 20, as a marker.

The one or more radiographic markers 30 may have a depth and be formed from the same material as the body 10. In some embodiments, the one or more radiographic markers 30 are embedded within the lattice structure 20 and characterized by portions of the lattice structure 20 being denser than the body 10. That is, the one or more radiographic markers 30 may be characterized by multiple layers of material laid on top of each other, thereby creating denser areas of material in association with the one or more radiographic markers 30. The one or more radiographic markers 30 may be printable within and supported by the lattice structure 20. The one or more radiographic markers 30 may be formed from the same material as the lattice structure 20. For example, the one or more radiographic markers 30 and the lattice structure 20 may be formed from grade 23 titanium alloy.

As seen most clearly in FIG. 14A, the one or more radiographic markers 30 may include a hollow channel 31 defined within the lattice structure 20 in the anterior region 13 of the implant 103. The hollow channel 31 may extend internally from an exterior surface or region 19 at the anterior region 13 of the body 10 towards an interior 27 of the body 10. In some embodiments, the one or more radiographic markers 30 include three (3) radiographic markers 30, where a first radiographic marker 30 includes a first hollow channel 31, a second radiographic marker 30 includes a second hollow channel 31, and a third radiographic marker 30 includes a third hollow channel 31 (see FIG. 14A). The second and third hollow channels 31 may also extend from an exterior 19 of the body 10 towards an interior 27 of the body 10.

In some configurations, the body 10 may have an interfixated design, such that screws or other fasteners may extend through the anterior region 13 of the body 10 into adjacent bone when the implant 100 is implanted within patient anatomy. The body may include channels 105 to receive screws or fasteners, the channels extending from the anterior region 13 to exit at a superior surface 11 and/or inferior surface 12, and/or through a portion of the anterior region 13. In other configurations, the body may receive screws or other fasteners for interfixation without providing specific channels.

The hollow channels 31 may be circular, elliptical, square, rectangular, or oblong. If the channels 31 are elliptical, rectangular, or oblong, they are positioned horizontally (e.g., along lateral edge 15 to lateral edge 16) or vertically (e.g., along superior surface 11 to inferior surface 12) within the body.

FIG. 15 illustrates a lateral end view of the implant 103 of FIGS. 13 through 14B, showing the rimless 42 nature of the body 10. Additionally illustrated are the convex edges 40 at the lateral edges 15, 16 of the body 10, as well as at the anterior region 13 and the posterior region 14 of the body 10. FIG. 15 additionally illustrates how the body 10 may taper towards the posterior region 14, such that the posterior region 14 of the body 10 is narrower than the anterior region 13 of the body 10.

Although not included in the embodiment illustrated in FIG. 15, radiographic markers could be placed in one or both of lateral edges 15 and 16. In such a situation, the radiographic markers may be similarly sized and shaped so as to visually align in a lateral radiographic image or may be slightly different in size and shape so as to each be visible in a lateral radiographic image.

### Cervical

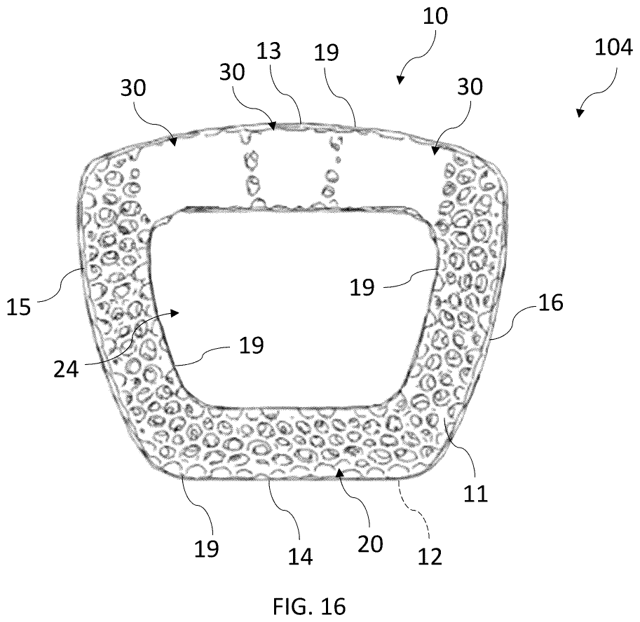

FIGS. 16 through 18 illustrate another embodiment of an implant 104. The implant 104 shares similar structures with the implant 100, the implant 102, and the implant 103, so like reference numerals will be used to refer to like elements and structures. Implant 104 may have features that are desirable in standard cervical fusion procedures. Similar to the implants 100, 102, and 103, the implant 104 includes a body 10 that has a lattice structure 20, where the body 10 and/or the lattice structure 20 defines a central void 24. The body 10 may be formed from and defined by the lattice structure 20. Specifically, the lattice structure 20 extends from a superior surface 11 of the body 10 to an inferior surface 12 of the body 10, and from a first lateral edge 15 to a second lateral edge 16. Additionally, the lattice structure 20 extends from an anterior region 13 to a posterior region 14.

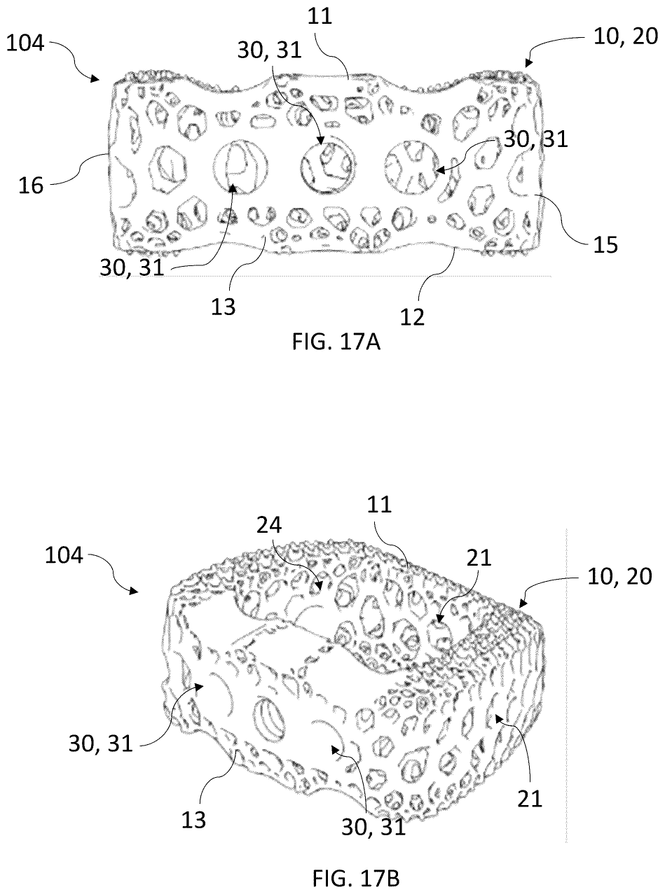

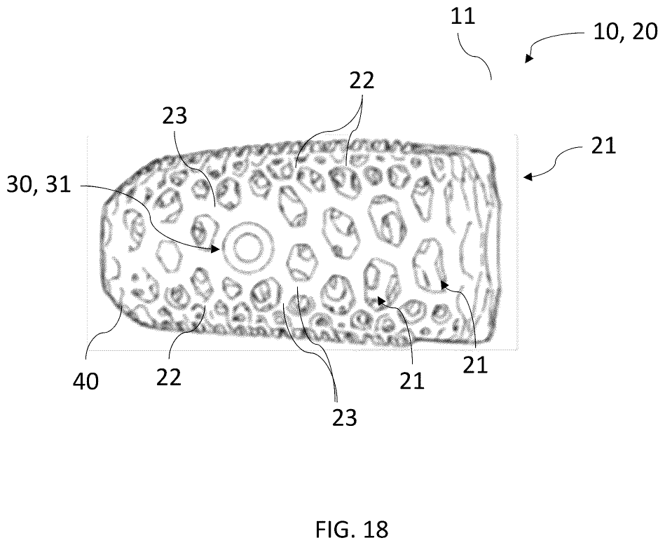

The lattice structure 20 may define the superior surface 11, the inferior surface 12, the first lateral edge 15, the second lateral edge 16, the anterior region 13, and the posterior region 14. That is, the lattice structure 20 defines the boundaries of the body 10. Additionally, referring briefly to FIGS. 17A through 18, the lattice structure 20 defines a plurality of pores 21. Specifically, each pore 21 of the plurality of pores 21 is defined by a plurality of vertices 22 and a plurality of beams 23. The number of vertices 22 and beams 23 defining each individual pore 21 may vary. Each pore 21 may be defined by four (4), five (5), six (6), seven (7), or more beams 23 and vertices 22. In general, the number of beams 23 defining the pores 21 corresponds to the number of vertices 22 defining the pores 21.

The lattice structure 20 may be a stochastic or substantially random lattice structure 20. In some embodiments, the lattice structure 20 is a mesh structure. As with the implants 100, 102, and 103, the lattice structure 20 may define a gradient of pore sizes as well as a gradient of beam diameters. As seen most clearly in FIG. 18, a size of pores 21 at the superior surface 11 may be smaller than a size of pores 21 in a middle region 18, between the superior surface 11 and the inferior surface 12. The size of pores 21 may become smaller again at the inferior surface 12. As each pore 21 is defined by a plurality of beams 23 and a plurality of vertices 22, and these beams 23 and vertices 22 are shared between pores 21, the transition between pore sizes is substantially continuous and seamless.

The size of pores 21 at the superior surface 11 and/or the inferior surface 12 may range from about 100 microns to about 1,000 microns, such as 200, 300, 350, 400, 450, 500, 550, 600, 650, 700, 750, 800, 850, 900, 950 microns, or a size within a range defined by any two of the foregoing values. Pores 21 having this size may be referred to as “micro pores.” The size of pores 21 in the middle region 18 may range from about 1,000 microns to about 8,000 microns, such as 1,500, 2,000, 2,500, 3,000, 3,500, 4,000, 4,500, 5,000, 5,500, 6,000, 6,500, 7,000, 7,500 microns, or a size within a range defined by any two of the foregoing values. Pores 21 having this size may be referred to as “macro pores.” The size of pores 21 at exterior regions 19 may be larger than a size of pores 21 at interior regions 27 of the lattice structure 20.

Additionally, the gradient of beam diameters follows or corresponds to the gradient of pore sizes. Specifically, a diameter of the beams 23 at the superior surface 11 and/or the inferior surface 12 may be smaller or narrower than a diameter of the beams 23 in the middle region 18. The defined gradient of pore sizes allows regions of a first pore size to transition to regions of a second pore size in a continuous and seamless manner. Specifically, as seen most clearly in FIG. 18, the pores 21 at the superior surface 11 share beams 23 with pores 21 in the middle region 18. Though the beam 23 diameter may change as the pores 21 transition from the superior surface 11 to the middle region 18, the transition between pore sizes is continuous (i.e., no clear boundaries or layers between the differing pore sizes). This increases the overall integrity of the implant 104.

The body 10 and/or the lattice structure 20 includes exterior regions 19 and interior regions 27. The exterior region 19 may correspond to boundaries of the lattice structure 20. For example, the lattice structure 20 defines the superior surface 11 and the inferior surface 12, where the superior surface 11 and the inferior surface 12 correspond to an exterior region or surface 19 of the implant 104. Similarly, the lattice structure 20 defines the central void 24 and boundaries of the central void 24 may correspond to exterior regions or surfaces 19. Likewise, areas or regions between or “inside” the superior surface 11, the exterior regions 19, and the inferior surface 12 may correspond to interior regions 27. In some embodiments, the exterior regions 19 correspond to surfaces of the body 10 that a user or practitioner may touch, while interior regions 27 are not tactilely accessible to the user or practitioner.

The gradient of pore sizes and beam diameters is also present from exterior regions 19 to interior regions 27. That is, a size of pores 21 at exterior regions 19 may be larger than a size of pores 21 at interior regions 27 of the lattice structure 20 and/or the body 10. Similarly, the beam diameters at exterior regions 19 may be larger than beam diameters at interior regions 27.

The variability and gradient of pore sizes and beam diameters achieves varying zones of stiffness between the superior and/or inferior surfaces 11, 12 as compared to an interior zone or region 27 of the body 10 and/or the lattice structure 20. This may result in the superior and/or inferior surfaces 11, 12 being more stiff than the interior regions 27; alternatively, the superior and/or inferior surfaces 11, 12 may be less stiff than the interior regions 27. Additionally, the variability of pore sizes and beam diameters achieves varying zones of stiffness between the exterior 19 of the body 10 as compared to an interior zone 27 of the body 10.

The body 10 and/or the lattice structure 20 additionally includes one or more radiographic markers 30. The one or more radiographic markers 30 allow practitioners to ensure proper placement and implantation of the implant 104 in a body of a subject. Specifically, the one or more radiographic markers 30 clearly mark the mid-line of the implant 104 when the implant 104 is properly positioned within the body of the subject, thereby providing a clear visualization that the implant 104 has been properly positioned. For example, the implant 104 may be placed within patient anatomy during a spinal surgical procedure. A radiographic marker 30 may be incorporated into the anterior region 13 and/or the posterior region 14 of the lattice structure 20 and/or the body 10. The one or more radiographic markers 30 may utilize improved radiolucency, relative to the rest of the lattice structure 20, as a marker.

The one or more radiographic markers 30 may have a depth and be formed from the same material as the body 10. In some embodiments, the one or more radiographic markers 30 are embedded within the lattice structure 20 and characterized by portions of the lattice structure 20 being denser than the body 10. That is, the one or more radiographic markers 30 may be characterized by multiple layers of material laid on top of each other, thereby creating denser areas of material in association with the one or more radiographic markers 30. The one or more radiographic markers 30 may be printable within and supported by the lattice structure 20. The one or more radiographic markers 30 may be formed from the same material as the lattice structure 20. For example, the one or more radiographic markers 30 and the lattice structure 20 may be formed from grade 23 titanium alloy.

As seen most clearly in FIG. 17A, the one or more radiographic markers 30 may include a hollow channel 31 defined within the lattice structure 20 in the anterior region 13 of the implant 103. The hollow channel 31 may extend internally from an exterior surface or region 19 at the anterior region 13 of the body 10 towards an interior 27 of the body 10. In some embodiments, the one or more radiographic markers 30 include three (3) radiographic markers 30, where a first radiographic marker 30 includes a first hollow channel 31, a second radiographic marker 30 includes a second hollow channel 31, and a third radiographic marker 30 includes a third hollow channel 31 (see FIG. 17A). The second and third hollow channels 31 may also extend from an exterior 19 of the body 10 towards an interior 27 of the body 10.

The hollow channels 31 may be circular, elliptical, square, rectangular, or oblong. If the channels 31 are elliptical, rectangular, or oblong, they are positioned horizontally (e.g., along lateral edge 15 to lateral edge 16) or vertically (e.g., along superior surface 11 to inferior surface 12) within the body.

The body 10 may have an interfixated design, such that screws or other fasteners may extend through the anterior region 13 of the body 10 into adjacent bone when the implant 103 is implanted within patient anatomy. The screws or fasteners may extend through a portion of the anterior region 13.

FIG. 18 illustrates a lateral end view of the implant 104 of FIGS. 16 through 17B. Similar to FIGS. 5, 11, and 15, FIG. 18 illustrates the rimless 42 nature of the body 10. Additionally illustrated are the convex edges 40 at the lateral edges 15, 16 of the body 10, as well as at the anterior region 13 and the posterior region 14 of the body 10. As before, being rimless 42 provides improved radiolucency to the implant 104 when the implant 104 is imaged through radiographic imaging, such as when ensuring proper placement of the implant 104 in patient anatomy.

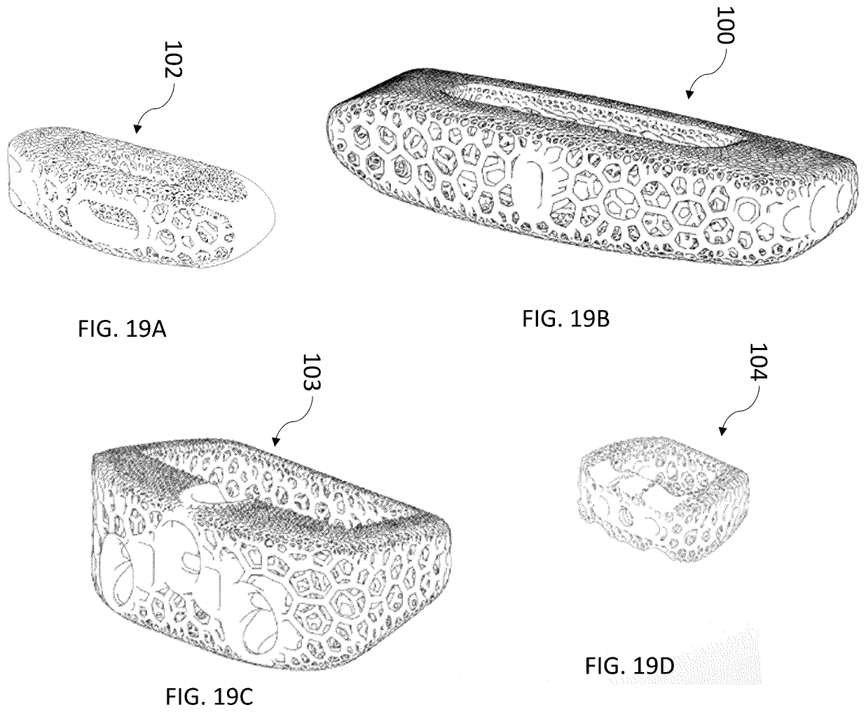

FIGS. 19A through 19D illustrate the implants of FIGS. 1, 7, 13, and 16. Each of these embodiments may be used for any type of surgical procedure as desired. The embodiment of FIG. 19A includes features that may be advantageous in a standard LIF spinal fusion procedure. The embodiment of FIG. 19B includes features that may be advantageous in a standard TLIF spinal fusion procedure. The embodiment of FIG. 19C includes features that may be advantageous in a standard ALIF spinal fusion procedure. The embodiment of FIG. 19D includes features that may be advantageous in a standard cervical fusion procedure.

FIG. 20 illustrates additional views of the implant 102 illustrated in FIGS. 7 through 11. The surface projections 25 can clearly be seen extending superiorly (and/or inferiorly) from the vertices 22 of the pores 21. Additionally, as illustrated, the radiographic marker 30 is circular or substantially circular.

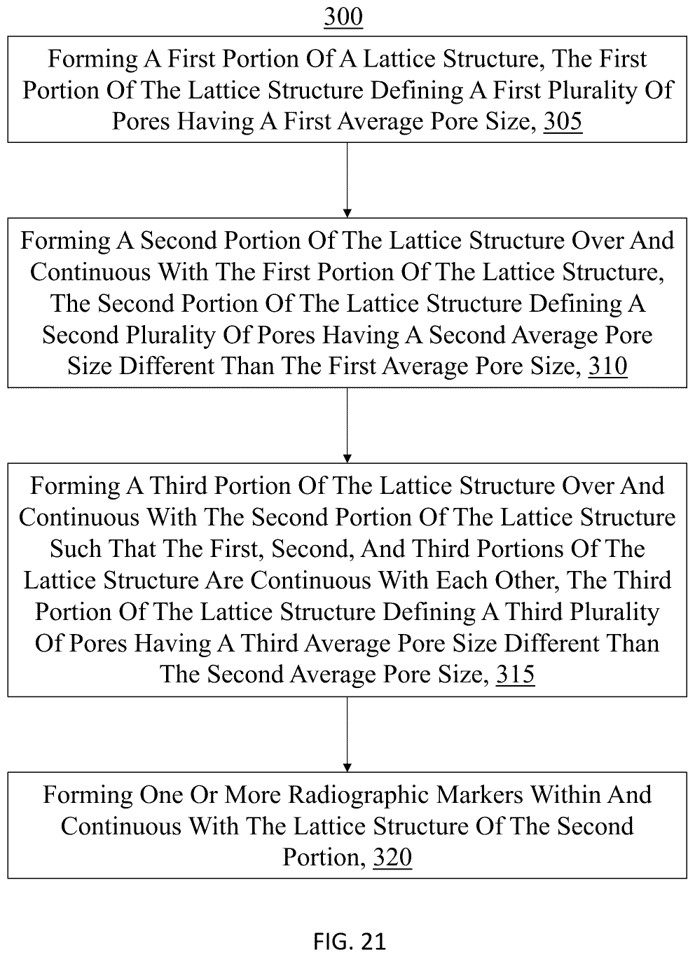

FIG. 21 is a flowchart of an example method 300 of manufacturing any one of the implants 100, 102, 103, and/or 104 from FIGS. 1 through 20. The method 300 may include forming a first portion of a lattice structure, the first portion of the lattice structure defining a first plurality of pores having a first average pore size, at 305. The first plurality of pores may be a plurality of micropores having a size ranging from 300 to 800 microns (μm). The method 300 may also include forming a second portion of the lattice structure over and continuous with the first portion of the lattice structure, the second portion of the lattice structure defining a second plurality of pores having a second average pore size different than the first average pore size, at 310. The second plurality of pores may be a plurality of macropores having a size ranging from 1 to 6 millimeters (mm).

Further, the method 300 may include forming a third portion of the lattice structure over and continuous with the second portion of the lattice structure such that the first, second, and third portions of the lattice structure are continuous with each other, the third portion of the lattice structure defining a third plurality of pores having a third average pore size different than the second average pore size, at 315. The third plurality of pores may be a plurality of micropores having a size ranging from 300 to 800 microns (μm). The second portion of the lattice structure may be more radiolucent than the first portion of the lattice structure. Still further, the method 300 may include forming one or more radiographic markers within and continuous with the lattice structure of the second portion, at 320.

The lattice structure may correspond to the lattice structure 20 of the implants 100, 102, 103, and 104. Forming the first, second, and/or third portions of the lattice structure may form a stochastic or mesh lattice structure. Additionally, forming the first, second, and/or third portions of the lattice structure may include forming a plurality of beams and a plurality of vertices, where the beams and the vertices at least partially define the first, second, and third plurality of pores. As described with respect to FIGS. 1 through 18, the plurality of beams 23 are continuous with each other and are shared among the plurality of pores 21. Accordingly, the first, second, and third portions of the lattice structure are continuous with each other with no borders or seams separating or defining the first, second, and/or third portions of the lattice structure.

The first portion of the lattice structure may form and define an inferior surface of the surgical implant. In some embodiments, the method 300 may additionally include disposing a plurality of projections about a portion of the inferior surface of the surgical implant, the plurality of projections having a directionality to reduce migration. The method 300 may also include varying a stiffness of the lattice structure of the first portion relative to the second portion. Varying a stiffness of the lattice structure may include forming a first plurality of beams of the lattice structure at an exterior region with a first thickness and forming a second plurality of beams of the lattice structure at an interior region with a second thickness, the second thickness being less than the first thickness, where the second plurality of beams are continuous with the first plurality of beams.

In some embodiments, forming the first, second, and/or third portions of the lattice structure includes printing the first, second, and/or third portions of the lattice structure. For example, the first and second portions may be printed using a 3D-printer printing with grade 23 titanium alloy.

In some embodiments, forming one or more radiographic markers within and continuous with the lattice structure includes embedding multiple layers of material within an anterior region of the second portion of the lattice structure, such that the one or more radiographic markers have a greater radiopacity than the immediately surrounding lattice structure, the material of the one or more radiographic markers being the same as the first, second, and third portions of the lattice structure. Additionally, and/or alternatively, forming one or more radiographic markers within and continuous with the lattice structure includes embedding multiple layers of material within a posterior region of the second portion of the lattice structure, such that the one or more radiographic markers have a greater radiopacity than the immediately surrounding lattice structure, the material of the one or more radiographic markers being the same as the first, second, and third portions of the lattice structure.

Additionally, and/or alternatively, forming one or more radiographic markers within and continuous with the lattice structure includes defining a first hollow channel within the lattice structure of the second portion in an anterior region of the lattice structure and defining a second hollow channel within a posterior region of the second portion of the lattice structure, such that the second hollow channel is visible through the first hollow channel when the surgical implant is properly placed and aligned during a surgical procedure. The first hollow channel may be larger than the second hollow channel. Either the first or second hollow channel may be circular in shape while the other channel is non-circular in shape. The non-circular shape may be oblong, rectangular, or elliptical.

Additionally, and/or alternatively, forming one or more radiographic markers within and continuous with the lattice structure includes embedding multiple layers of material within a proximal region of the second portion of the lattice structure, such that the one or more radiographic markers have a greater radiopacity than the immediately surrounding lattice structure, the material of the one or more radiographic markers being the same as the first, second, and third portions of the lattice structure.

The one or more radiographic markers of the posterior region may be positioned relative to the one or more radiographic markers of the anterior region so as to provide a visual indication of alignment when implanted into a patient's anatomy.

In some embodiments, the method 300 includes forming one or more holes or shafts in at least one of the first, second, and third lattice structures and inserting one or more second radiographic markers into the one or more holes or shafts. The one or more second radiographic markers comprise a material distinct from the material of the lattice structure.

### Embodiments

The following embodiments are specific examples of what has been contemplated by the authors of this disclosure:

Embodiment 1. A surgical implant comprising:
a body comprising a lattice structure, the lattice structure extending from a superior surface of the body to an inferior surface of the body and a first lateral edge to a second lateral edge, the lattice structure defining (i) the superior surface and the inferior surface of the body, (ii) a central void of the body, (iii) a gradient of pore sizes, and (iv) a gradient of beam diameters,
both beam diameter and pore size varying from the superior and inferior surfaces of the body and from exterior to interior surfaces of the body;
a plurality of superior surface projections extending superiorly from a portion of the lattice structure at the superior surface, each of the plurality of superior surface projections having a directionality for reducing migration of the surgical implant after placement of the surgical implant during a surgical procedure; and
one or more radiographic markers incorporated into the body.

Embodiment 2. The surgical implant of embodiment 1, wherein the variability of pore sizes and beam diameters achieves varying zones of stiffness between the superior and/or inferior surfaces as compared to an interior zone of the body.

Embodiment 3. The surgical implant of embodiment 2, wherein the superior and/or inferior surfaces are less stiff than the interior zone.

Embodiment 4. The surgical implant of embodiment 2, wherein the superior and/or inferior surfaces are stiffer than the interior zone.

Embodiment 5. The surgical implant of any one of embodiments 1 through 3, wherein the variability of pore sizes and beam diameters achieves varying zones of stiffness between the exterior of the body as compared to an interior zone of the body.

Embodiment 6. The surgical implant of embodiment 5, wherein the exterior is stiffer than the interior zone.

Embodiment 7. The surgical implant of embodiment 5, wherein the exterior is less stiff than the interior zone.

Embodiment 8. The surgical implant of any one of embodiments 1 through 7, wherein the plurality of superior surface projections are disposed radially about the central void of the body and extend from the central void to a first boundary of the body.

Embodiment 9. The surgical implant of any one of embodiments 1 through 7, wherein the plurality of superior surface projections are disposed radially relative to a proximal end of the body and extend toward a distal end of the body.

Embodiment 10. The surgical implant of any one of embodiments 1 through 7, wherein the plurality of superior surface projections are disposed radially relative to a distal end of the body and extend toward a proximal end of the body.

Embodiment 11. The surgical implant of embodiment 10, wherein the radial pattern of the projections is configured to facilitate rotation of the interbody about a distal pivot point when implanted in an intervertebral disc space.

Embodiment 12. The surgical implant of any one of embodiments 1 through 11, wherein the directionality of the plurality of superior surface projections is toward a proximal end of the body.

Embodiment 13. The surgical implant of any one of embodiments 1 through 12, further comprising a plurality of inferior surface projections extending inferiorly from a portion of the lattice structure at the inferior surface of the body, each of the plurality of inferior projections having a directionality for reducing migration of the surgical implant after placement of the surgical implant during a surgical procedure.

Embodiment 14. The surgical implant of embodiment 13, wherein the directionality of the inferior surface projections is similar to the directionality of the superior surface projections.

Embodiment 15. The surgical implant of any one of embodiments 1 through 14, wherein each of the plurality of superior and/or inferior surface projections are located at respective vertices of the pores.

Embodiment 16. The surgical implant of any one of embodiments 1 through 15, wherein the superior and/or inferior surface projections exhibit varying heights with taller surface projections located near the central void and shorter surface projections located toward the first and second lateral edges.

Embodiment 17. The surgical implant of any one of embodiments 1 through 16, wherein the superior and/or inferior surface projections are grouped into a plurality of bands extending across the superior and/or inferior surfaces from the first lateral edge to the second lateral edge.

Embodiment 18. The surgical implant of any one of embodiments 1 through 17, wherein the gradient of pores comprises (i) one or more regions of micropores beginning at the superior surface and having sizes ranging from 100 microns to 1,000 microns, (ii) one or more regions of macropores in the internal portion of the lattice structure, the one or more regions of macropores having sizes ranging from 1,000 microns to 8,000 microns, and (iii) one or more regions of micropores at the inferior surface.

Embodiment 19. The surgical implant of embodiment 18, wherein the micropores of the superior surface range in size from 300 microns to 800 microns and the macropores of the internal portion range in size from 1,000 microns to 6,000 microns.

Embodiment 20. The surgical implant of either of embodiments 18 or 19, wherein the one or more regions of micropores transition continuously to the one or more regions of macropores.

Embodiment 21. The surgical implant of any one of embodiments 1 through 20, wherein the lattice structure is a stochastic lattice structure.

Embodiment 22. The surgical implant of any one of embodiments 1 through 20, wherein the lattice structure is a mesh structure.

Embodiment 23. The surgical implant of any one of embodiments 1 through 22, further comprising a plurality of teeth disposed about edges of the superior surface of the body, the plurality of teeth being disposed along a line of curvature to facilitate rotational placement of the surgical implant during a surgical procedure.

Embodiment 24. The surgical implant of any one of embodiments 1 through 23, wherein the one or more radiographic markers comprise a first hollow channel within the lattice structure, the hollow channel extending internally from an exterior surface of the body towards an interior of the body.

Embodiment 25. The surgical implant of embodiment 24, wherein the one or more radiographic markers comprise a second hollow channel within the lattice structure, the hollow channel extending internally from an exterior surface of the body towards an interior of the body, the second hollow channel configured to align with the first hollow channel as an indication of desired positioning of the surgical implant within a patient's anatomy.

Embodiment 26. The surgical implant of either of embodiments 24 or 25, wherein the first and/or second hollow channel is circular, elliptical, square, rectangular, or oblong.

Embodiment 27. The surgical implant of embodiment 26, wherein the first and/or second hollow channel, if elliptical, rectangular, or oblong, is positioned horizontally or vertically within the body.

Embodiment 28. The surgical implant of any one of embodiments 22 through 27, wherein one of the first or second hollow channels is circular and the other is non-circular.

Embodiment 29. The surgical implant of embodiment 28, wherein the first hollow channel is circular and the second hollow channel is oblong.

Embodiment 30. The surgical implant of any one of embodiments 1 through 29, wherein at least one of the one or more radiographic markers utilizes improved radiolucency as a marker.

Embodiment 31. The surgical implant of any one of embodiments 1 through 30, wherein the one or more radiographic markers extend from an external edge of the body towards an interior of the body, the one or more radiographic markers having a depth, and the one or more radiographic markers being formed from the same material as the body.

Embodiment 32. The surgical implant of any one of embodiments 1 through 31, wherein the one or more radiographic markers are embedded within the lattice structure and characterized by portions of the lattice structure being denser than the body.

Embodiment 33. The surgical implant of any one of embodiments 1 through 32, wherein the one or more radiographic markers are printable within and supported by the lattice structure.

Embodiment 34. The surgical implant of any one of embodiments 1 through 33, wherein the lateral edges of the superior and inferior surfaces are rounded.

Embodiment 35. The surgical implant of any one of embodiments 1 through 34, further comprising an anterior surface that defines a convex curve.

Embodiment 36. The surgical implant of embodiment 35, wherein the superior and inferior surfaces define respective planes that converge at a point posterior to the anterior surface.

Embodiment 37. The surgical implant of any one of embodiments 1 through 36, further comprising an inserter engagement portion comprising an interior space accessible through an elongated window in a proximal end of the body, wherein the elongated window comprises a plurality of raised surface features configured to engage with a portion of the inserter, and wherein the interior space is cylindrical.

Embodiment 38. An implant for use in spinal surgical procedures, the implant comprising:
a body comprising a lattice structure, the lattice structure extending continuously from a superior surface of the body to an inferior surface of the body,
the lattice structure defining a gradient of pores, each pore in the gradient having a plurality of vertices;
a plurality of projections extending superiorly from each of the plurality of vertices of each pore of the gradient of pores at the superior surface of the body, each projection of the plurality of projections having a directionality; and
one or more radiographic markers incorporated into the lattice structure, the one or more radiographic markers having a density greater than the density of the lattice structure immediately around the one or more radiographic markers and being comprised of the same material as the lattice structure.

Embodiment 39. The implant of embodiment 38, wherein the gradient of pores comprises a first plurality of micropores at or near the superior surface of the body, a plurality of macropores at or near a center of the body, and a second plurality of micropores at or near the inferior surface of the body.

Embodiment 40. The implant of embodiment 39, wherein the first plurality of micropores has a size ranging from 300 to 800 microns (μm).

Embodiment 41. The implant of either one of embodiments 39 or 40, wherein the plurality of macropores has a size ranging from 1 to 6 millimeters (mm).

Embodiment 42. The implant of any one of embodiments 38 through 41, wherein the second plurality of micropores have a size ranging from 500 to 700 microns (μm).

Embodiment 43. The implant of any one of embodiments 38 through 42, wherein the gradient of pores continuously transitions from the first plurality of micropores to the plurality of macropores and to the second plurality of micropores.

Embodiment 44. The implant of any one of embodiments 38 through 43, wherein the lattice structure comprises a plurality of beams defining the gradient of pores and wherein an internal plurality of beams has a diameter less than an external plurality of beams.

Embodiment 45. The implant of any one of embodiments 38 through 44, wherein the body is a rimless body, such that a boundary of the superior surface of the body is defined by the plurality of projections at the superior surface.

Embodiment 46. The implant of any one of embodiments 38 through 45, further comprising a second plurality of projections, each of the second plurality of projections extending inferiorly from at least some of the vertices defining the pores at the inferior surface of the body, each projection of the second plurality of projections having a directionality.

Embodiment 47. The implant of embodiment 46, wherein the body is a rimless body, such that a boundary of the inferior surface of the body is defined by the second plurality of projections at the inferior surface.

Embodiment 48. A spinal implant device comprising:
a body comprising a lattice structure;
a plurality of pores defined by the lattice structure, a first group of pores defined at a superior surface of the body, each pore of the first group of pores defined by a plurality of vertices; and
a plurality of projections extending superiorly from at least some of the plurality of vertices of the first group of pores, each projection of the plurality of projections having a directionality to reduce spinal implant migration.

Embodiment 49. The spinal implant device of embodiment 48, further comprising a second group of pores defined at an inferior surface of the body, each pore of the second group of pores defining a plurality of vertices.

Embodiment 50. The spinal implant device of embodiment 49, further comprising a second plurality of projections extending inferiorly from at least some of the plurality of vertices of the second group of pores, each projection of the second plurality of projections having a directionality.

Embodiment 51. The spinal implant device of embodiment 50, wherein the directionality of the second plurality of projections and the directionality of the plurality of projections are the same.

Embodiment 52. The spinal implant device of any one of embodiments 44 through 51, wherein the lattice structure comprises an internal lattice structure and an external lattice structure, the external lattice structure having a beam diameter greater than a beam diameter of the internal lattice structure.

Embodiment 53. The spinal implant device of any one of embodiments 45 through 52, wherein the first and second plurality of projections form a repeating pattern that extends radially from a central void defined by the body to a first boundary.

Embodiment 54. A method of manufacturing a surgical implant comprising:
forming a first portion of a lattice structure, the first portion of the lattice structure defining a first plurality of pores having a first average pore size;
forming a second portion of the lattice structure over and continuous with the first portion of the lattice structure, the second portion of the lattice structure defining a second plurality of pores having a second average pore size different than the first average pore size;
forming a third portion of the lattice structure over and continuous with the second portion of the lattice structure such that the first, second, and third portions of the lattice structure are continuous with each other, the third portion of the lattice structure defining a third plurality of pores having a third average pore size different than the second average pore size;
the second portion being more radiolucent than the first portion;
forming one or more radiographic markers within and continuous with the lattice structure of the second portion.

Embodiment 55. The method of embodiment 54, wherein the first portion of the lattice structure forms and defines an inferior surface of the surgical implant.

Embodiment 56. The method of embodiment 55, further comprising disposing a plurality of projections about a portion of the inferior surface of the surgical implant, the plurality of projections having a directionality to reduce migration.

Embodiment 57. The method of any one of embodiments 52 through 56, further comprising varying a stiffness of the lattice structure of the first portion relative to the second portion.

Embodiment 58. The method of embodiment 57, wherein varying the stiffness of the lattice structure comprises:
forming a first plurality of beams of the lattice structure at an exterior region with a first thickness; and
forming a second plurality of beams of the lattice structure at an interior region with a second thickness, the second thickness being less than the first thickness,
the second plurality of beams being continuous with the first plurality of beams.

Embodiment 59. The method of any one of embodiments 54 through 58, wherein forming a first portion of a lattice structure comprises printing the first portion of the lattice structure.

Embodiment 60. The method of any one of embodiments 54 through 59, wherein forming a second portion of a lattice structure comprises printing the second portion of the lattice structure.

Embodiment 61. The method of any one of embodiments 54 through 60, wherein forming a third portion of a lattice structure comprises printing the third portion of the lattice structure.

Embodiment 62. The method of any one of embodiments 54 through 61, wherein forming one or more radiographic markers within and continuous with the lattice structure comprises embedding multiple layers of material within an anterior region of the second portion of the lattice structure, such that the one or more radiographic markers have a greater radiopacity than the immediately surrounding lattice structure, the material of the one or more radiographic markers being the same as the first, second, and third portions of the lattice structure.

Embodiment 63. The method of any one of embodiments 54 through 62, wherein forming one or more radiographic markers within and continuous with the lattice structure comprises embedding multiple layers of material within a posterior region of the second portion of the lattice structure, such that the one or more radiographic markers have a greater radiopacity than the immediately surrounding lattice structure, the material of the one or more radiographic markers being the same as the first, second, and third portions of the lattice structure.

Embodiment 64. The method of embodiment 63, wherein the one or more radiographic markers of the posterior region are positioned relative to the one or more radiographic markers of the anterior region so as to provide a visual indication of alignment when implanted into a patient's anatomy.

Embodiment 65. The method of any one of embodiments 54 through 64, wherein forming one or more radiographic markers within and continuous with the lattice structure comprises:
defining a first hollow channel within the lattice structure of the second portion in an anterior region of the lattice structure; and
defining a second hollow channel within a posterior region of the second portion of the lattice structure, such that the second hollow channel is visible through the first hollow channel when the surgical implant is properly placed and aligned during a surgical procedure.

Embodiment 66. The method of embodiment 65, wherein the first hollow channel is larger than the second hollow channel.

Embodiment 67. The method of either one of embodiments 65 or 66, wherein either the first or second hollow channel is circular in shape and the other channel is non-circular in shape.

Embodiment 68. The method of embodiment 67, wherein the non-circular shape is one of oblong, rectangular, or elliptical.

Embodiment 69. The method of any one of embodiments 54 through 68, wherein forming one or more radiographic markers within and continuous with the lattice structure comprises embedding multiple layers of material within a proximal region of the second portion of the lattice structure, such that the one or more radiographic markers have a greater radiopacity than the immediately surrounding lattice structure, the material of the one or more radiographic markers being the same as the first, second, and third portions of the lattice structure.

Embodiment 70. The method of any one of embodiments 54 through 69, further comprising forming one or more holes or shafts in at least one of the first, second, and third lattice structures and inserting one or more second radiographic markers into the one or more holes or shafts.

Embodiment 71. The method of embodiment 70, wherein the one or more second radiographic markers comprise a material distinct from the material of the lattice structure.

Embodiment 72. A method of inserting the porous interbody of any one of embodiments 1 through 53, the method comprising:
advancing the interbody along a surgical approach to an intervertebral disc space, the interbody releasably secured to an inserter, the inserter defining a first axis, and the interbody having a longitudinal length substantially aligned with the first axis as the interbody is advanced into the intervertebral disc space;
positioning a distal end of the interbody in a first desired location of the intervertebral disc space, the first desired location being substantially anterior within the intervertebral disc space and toward a lateral side of the intervertebral disc space;
manipulating the inserter to allow the interbody to pivot relative to a distal end of the inserter;
positioning a proximal end of the interbody in a second desired location of the intervertebral disc space, the second desired location being substantially anterior within the intervertebral disc space and toward
wherein positioning the proximal end of the interbody in the second desired location is achieved by laterally translating with the inserter the proximal end of the interbody with the interbody pivoting about the distal end, such that as the interbody pivots about the interbody distal end, the interbody simultaneously pivots relative to the inserter at the interbody proximal end.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular surgical implant (e.g., a spinal interbody implant); however, other implants are also contemplated.

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expect skilled artisans to employ such variations as appropriate, and the author(s) intend for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A surgical implant comprising:
a body comprising a lattice structure, the lattice structure extending from a superior surface of the body to an inferior surface of the body and a first lateral edge to a second lateral edge, the lattice structure defining (i) the superior surface and the inferior surface of the body, (ii) a central void of the body, (iii) a gradient of pore sizes, and (iv) a gradient of beam diameters,
both beam diameter and pore size varying from the superior and inferior surfaces of the body and from exterior to interior surfaces of the body;
a plurality of superior surface projections extending superiorly from a portion of the lattice structure at the superior surface, each of the plurality of superior surface projections having a directionality for reducing migration of the surgical implant after placement of the surgical implant during a surgical procedure; and
one or more radiographic markers incorporated into the body.

2. The surgical implant of claim 1, wherein the variability of pore sizes and beam diameters achieves varying zones of stiffness between the superior and/or inferior surfaces as compared to an interior zone of the body.

3. The surgical implant of claim 2, wherein the superior and/or inferior surfaces are less stiff than the interior zone.

4. The surgical implant of claim 2, wherein the superior and/or inferior surfaces are stiffer than the interior zone.

5. The surgical implant of claim 1, wherein the variability of pore sizes and beam diameters achieves varying zones of stiffness between the exterior of the body as compared to an interior zone of the body.

6. The surgical implant of claim 5, wherein the exterior is stiffer than the interior zone.

7. The surgical implant of claim 5, wherein the exterior is less stiff than the interior zone.

8. The surgical implant of claim 1, wherein the plurality of superior surface projections are disposed radially about the central void of the body and extend from the central void to a first boundary of the body.

9. The surgical implant of claim 1, wherein the plurality of superior surface projections are disposed radially relative to a proximal end of the body and extend toward a distal end of the body.

10. The surgical implant of claim 1, wherein the plurality of superior surface projections are disposed radially relative to a distal end of the body, forming a radial pattern, and wherein the plurality of superior surface projections extend toward a proximal end of the body.

11. The surgical implant of claim 10, wherein the radial pattern of the projections is configured to facilitate rotation of the implant about a distal pivot point when implanted in an intervertebral disc space.

12. The surgical implant of claim 1, wherein the directionality of the plurality of superior surface projections is toward a proximal end of the body.

13. The surgical implant of claim 1, wherein each of the plurality of superior and/or inferior surface projections is located at a respective vertex of the pores.

14. The surgical implant of claim 1, wherein the superior and/or inferior surface projections exhibit varying heights with taller surface projections located near the central void and shorter surface projections located toward the first and second lateral edges.

15. The surgical implant of claim 1, wherein the superior and/or inferior surface projections are grouped into a plurality of bands extending across the superior and/or inferior surfaces from the first lateral edge to the second lateral edge.

16. The surgical implant of claim 1, wherein the gradient of pores comprises (i) one or more regions of micropores beginning at the superior surface and having sizes ranging from 100 microns to 1,000 microns, (ii) one or more regions of macropores in an internal portion of the lattice structure, the one or more regions of macropores having sizes ranging from 1,000 microns to 8,000 microns, and (iii) one or more regions of micropores at the inferior surface.

17. The surgical implant of claim 16, wherein the micropores of the superior surface range in size from 300 microns to 800 microns and the macropores of the internal portion range in size from 1,000 microns to 6,000 microns.

18. The surgical implant of either of claim 17, wherein the one or more regions of micropores transition continuously to the one or more regions of macropores.

19. The surgical implant of claim 1, wherein the lattice structure is a stochastic lattice structure.

20. The surgical implant of claim 1, wherein the lattice structure is a mesh structure.
